(19) United States
(12) Patent Application Publication
(10) Pub. No.: US 2015/0297564 A1
(43) Pub. Date: Oct. 22, 2015

(54) METHOD OF ENHANCING GLUCOSE-STIMULATED INSULIN SECRETION AND OF TREATING TYPE 2 DIABETES OR HYPOGLYCEMIA

(71) Applicant: Xingen Lei, Ithaca, NY (US)

(72) Inventor: Xingen Lei, Ithaca, NY (US)

(73) Assignee: CORNELL UNIVERSITY, Ithaca, NY (US)

(21) Appl. No.: 14/691,213

(22) Filed: Apr. 20, 2015

Related U.S. Application Data
(60) Provisional application No. 61/981,301, filed on Apr. 18, 2014.

(51) Int. Cl. A61K 31/41; A61K 38/44

(52) U.S. Cl. CPC A61K 31/41; A61K 38/44; C12Y 111/01009

(57) ABSTRACT

The present invention is directed to a method of enhancing glucose-stimulated insulin secretion in a subject. The method involves selecting a subject with: (1) an antioxidant deficiency and (2) a need for enhanced glucose-stimulated insulin secretion, and administering to the selected subject an agent selected from the group consisting of (1) a compound according to Formula I or a pharmaceutically acceptable salt thereof:

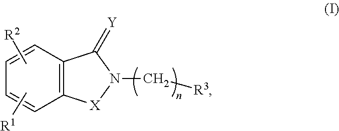

wherein the substituents R¹-R³, X, Y, and n are as described herein, (2) glutathione peroxidase, or (3) activators of PGC-1α antioxidant response element, under conditions effective to enhance glucose-stimulated insulin secretion in the subject. The present invention also relates to methods of treating a subject with Type 2 diabetes and treating a subject with hypoglycemia by administering these agents.

This application claims the benefit of U.S. Provisional Patent Application Ser. No. 61/981,301, filed Apr. 18, 2014, which is hereby incorporated by reference in its entirety.

This invention was made with government support under National Institute of Health grant number DK53018. The government has certain rights in this invention.

### FIELD OF THE INVENTION

The present invention relates to a method of enhancing glucose-stimulated insulin secretion and of treating Type 2 diabetes or hypoglycemia.

### BACKGROUND OF THE INVENTION

Type 2 diabetes is one of the most prevalent chronic diseases worldwide. While insulin resistance was regarded as a hallmark of this disease, defective insulin secretion has recently been recognized as the main culprit (Ashcroft et al., “Diabetes Mellitus and the Beta Cell: The Last Ten Years,” Cell 148:1160-1171 (2012)). Being a “privilege” of aerobic organisms, oxidative stress is implicated in glucose-stimulated insulin secretion (“GSIS”) of pancreatic islet β-cells (Pi et al., “ROS Signaling, Oxidative Stress and Nrf2 in Pancreatic Beta-Cell Function,” Toxicol. Appl. Pharmacol. 244:77-83 (2009)). Thus, the relatively low expression of antioxidant enzymes in islets (Lenzen et al., “Low Antioxidant Enzyme Gene Expression in Pancreatic Islets Compared with Various Other Mouse Tissues,” Free Radic. Biol. Med. 20:463-466 (1996)) may not only render them susceptible to oxidative insults, but also provide a necessary metabolic condition for their sensitive responses to reactive oxygen species (“ROS”)-mediated signaling in GSIS (Pi et al., “Reactive Oxygen Species as a Signal in Glucose-Stimulated Insulin Secretion,” Diabetes 56:1783-1791 (2007)). In fact, H₂O₂ functions as an essential second messenger (Forman, H. J., “Reactive Oxygen Species and Alpha, Beta-Unsaturated Aldehydes as Second Messengers in Signal Transduction,” Ann. N Y Acad. Sci. 1203:35-44 (2010)) in initiating and regulating GSIS (Pi et al., “Reactive Oxygen Species as a Signal in Glucose-Stimulated Insulin Secretion,” Diabetes 56:1783-1791 (2007); Pi et al., “ROS Signaling, Oxidative Stress and Nrf2 in Pancreatic Beta-Cell Function,” Toxicol. Appl. Pharmacol. 244:77-83 (2009)), and demonstrates concentration-dependent dual effects on insulin signaling and other metabolic processes (Iwakami et al., “Concentration-Dependent Dual Effects of Hydrogen Peroxide on Insulin Signal Transduction in H4IIEC Hepatocytes,” PLoS One 6:e27401 (2011); Piwkowska et al., “Hydrogen Peroxide Induces Activation of Insulin Signaling Pathway via AMP-Dependent Kinase in Podocytes,” Biochem. Biophys. Res. Commun. 428:167-172 (2012)).

Se-dependent glutathione peroxidase-1 (“GPX1”) and Cu,Zn-superoxide dismutase (“SOD1”) represent two major intracellular antioxidant enzymes that can modulate intracellular H₂O₂ status. Despite low GPX1 and SOD1 activities in islets (only 2% and 29% of that in liver, respectively) (Lenzen et al., “Low Antioxidant Enzyme Gene Expression in Pancreatic Islets Compared with Various Other Mouse Tissues,” Free Radic. Biol. Med. 20:463-466 (1996)), it was found that knockout of GPX1 (“GKO”) and SOD1 (“SKO”) alone or in combination (“dKO”) caused substantial impairment of GSIS (Wang et al., “Knockouts of SOD1 and GPX1 Exert Different Impacts on Murine Islet Function and Pancreatic Integrity,” Antioxid. Redox Signal. 14:391-401 (2011)). Consistently, a Gpx1 gene variant (C198T) lowering the enzyme activity was identified in the South Indian population, which resulted in increased incidences of type 2 diabetes (C/T, 1.4-fold and T/T, 1.8-fold) (Ramprasath et al., “Genetic Association of Glutathione Peroxidase-1 (GPx-1) and NAD(P)H:Quinone Oxidoreductase 1 (NQ01) Variants and Their Association of CAD in Patients with Type-2 Diabetes,” Mol. Cell. Biochem. 361:143-150 (2012)). Intriguingly, the overt phenotypes of GSIS impairments in the GKO and SKO mice were similar (Wang et al., “Knockouts of SOD1 and GPX1 Exert Different Impacts on Murine Islet Function and Pancreatic Integrity,” Antioxid. Redox Signal. 14:391-401 (2011)), although GPX1 catalyzes H₂O₂ breakdown whereas SOD1 catalyzes H₂O₂ formation. It is puzzling that the presumed opposite effects of these two knockouts on islet intracellular H₂O₂ production might have induced seemingly similar biochemical and signaling regulation of GSIS. The biochemical regulation of GSIS depends on four key proteins: glucose transporter type 2 (“GLUT2”), glucokinase (“GK”), pancreatic and duodenal homeobox 1 (“PDX1”), and uncoupling protein 2 (“UCP2”) (Jensen et al., “Metabolic Cycling in Control of Glucose-Stimulated Insulin Secretion,” Am. J. Physiol. Endocrinol. Metab. 295:E1287-1297 (2008)). However, it remains unclear if the impacts of GKO and SKO on GSIS were mediated by altering functional expressions of these proteins in a coordinated fashion. Although changes of PDX1 and UCP2 (Wang et al., “Knockouts of SOD1 and GPX1 Exert Different Impacts on Murine Islet Function and Pancreatic Integrity,” Antioxid. Redox Signal. 14:391-401 (2011)) were previously observed in the whole pancreas of these knockout mice, systematic responses of GK, GLUT2, PDX1, and UCP2 in their islets have not been studied. More importantly, there is no information on the signaling cascade and molecular mechanism to link these knockout-initiated islet intracellular ROS changes to the responses of these four proteins and the observed GSIS phenotypes.

The gene promoter regions of GK, GLUT2, PDX1, and UCP2 may share common domains that bind transcriptional factors involved in signaling pathways related to the above-described question (Mazzarelli et al., “EPConDB: A Web Resource for Gene Expression Related to Pancreatic Development, Beta-Cell Function and Diabetes,” Nucleic Acids Res. 35:D751-D755 (2007)). The first is the peroxisome proliferator-activated receptor gamma coactivator 1 alpha (“PGC-1α”)-mediated antioxidant response element (“ARE”) signaling pathway. While PGC-1α is involved in responses of various genes to redox regulation (Tkachev et al., “Mechanism of the Nrf2/Keap1/ARE Signaling System,” Biochemistry (Mosc.) 76:407-422 (2011)) and regulates GSIS in human islets (Ling et al., “Epigenetic Regulation of PPARGC1A in Human Type 2 Diabetic Islets and Effect on Insulin Secretion,” Diabetologia 51:615-622 (2008)), two of its gene variants are associated with increased risks of type 2 diabetes in the Indian population (Bhat et al., “PGC-1 alpha Thr394Thr and Gly482Ser Variants are Significantly Associated with T2DM in Two North Indian Populations: A Replicate Case-Control Study,” Hum. Genet. 121:609-614 (2007); Yang et al., “Association of Peroxisome Proliferator-Activated Receptor Gamma Coactivator 1 Alpha (PPARGC1A) Gene Polymorphisms and Type 2 Diabetes Mellitus: A Meta-Analysis,” Diabetes Metab. Res. Rev. 27:177-184 (2011)). The second is the glucocorticoid receptor (“GR”) pathway that is negatively regulated by ROS or H₂O₂ in inflammation and immune responses (Kao et al., “Glycyrrhizic Acid and 18beta-Glycyrrhetinic Acid Recover Glucocorticoid Resistance via PI3K-Induced API, CRE and NFAT Activation,” Phytomedicine 20:295-302 (2013)). Polymorphisms of gr are associated with type 2 diabetes with low insulin levels and attenuated first phase of GSIS in women (van Raalte et al., “Glucocorticoid Receptor Gene Polymorphisms are Associated with Reduced First-Phase Glucose-Stimulated Insulin Secretion and Disposition Index in Women, but not in Men,” Diabet. Med. 29:e211-e216 (2012)). The third is the Wnt pathway that is positively regulated by ROS or H₂O₂ (Wen et al., “Reactive Oxygen Species and Wnt Signalling Crosstalk Patterns Mouse Extraembryonic Endoderm,” Cell. Signal. 24:2337-2348 (2012)). Elevated oxidation status by selenium deficiency (Kipp et al., “Four Selenoproteins, Protein Biosynthesis, and Wnt Signalling are Particularly Sensitive to Limited Selenium Intake in Mouse Colon,” Mol. Nutr. Food Res. 53:1561-1572 (2009)) or Gpx3 knockout (Barrett et al., “Tumor Suppressor Function of the Plasma Glutathione Peroxidase gpx3 in Colitis-Associated Carcinoma,” Cancer Res. 73:1245-1255 (2013)) activated the Wnt pathway in mice. This pathway activated gk gene transcription and insulin secretion in isolated islets of C57BL/6 mice (Schinner et al., “Regulation of Insulin Secretion, Glucokinase Gene Transcription and Beta Cell Proliferation by Adipocyte-Derived Wnt Signalling Molecules,” Diabetologia 51:147-154 (2008)), and has at least four variants of TCF7L2 associated with increased risks of type 2 diabetes (Tong et al., “Association Between TCF7L2 Gene Polymorphisms and Susceptibility to Type 2 Diabetes Mellitus: A Large Human Genome Epidemiology (HuGE) Review and Meta-Analysis,” BMC Med. Genet. 10:15 (2009)). The fourth is the NFAT pathway that stimulates gk, glut2, and pdx1 expression and insulin secretion in β-cell (Heit et al., “Calcineurin/NFAT Signalling Regulates Pancreatic Beta-Cell Growth and Function,” Nature 443:345-349 (2006)). This pathway in mouse C141 cells is activated by superoxide, but inhibited by H₂O₂ (Ke et al., “Essential Role of ROS-Mediated NFAT Activation in TNF-Alpha Induction by Crystalline Silica Exposure,” Am. J. Physiol. Lung Cell. Mol. Physiol. 291:L257-L264 (2006)). Because many members in these four signaling pathways are ROS-responsive and involved in GSIS, and can bind to domains in the gene promoter regions of GLUT2, GK, PDX1, and UCP2, it is fascinating to explore if GKO, SKO, and dKO initiated their impact on GSIS via these pathways.

The GPX mimic ebselen has been shown to protect against oxidative injuries (Day, B. J., “Catalase and Glutathione Peroxidase Mimics. Biochem. Pharmacol. 77:285-296 (2009)), suppress (Costa et al., “Ebselen Reduces Hyperglycemia Temporarily-Induced by Diazinon: A Compound with Insulin-Mimetic Properties,” Chem. Biol. Interact. 197:80-86 (2012)) the diazinon-induced hyperglycemia in rats, and decrease islet UCP2 (Wang et al., “Molecular Mechanisms for Hyperinsulinaemia Induced by Overproduction of Selenium-Dependent Glutathione Peroxidase-1 in Mice,” Diabetologia 51:1515-1524 (2008)). The SOD mimic copper diisopropylsalicylate (CuDIPs) attenuated the streptozotocin (STZ)-induced diabetes in rats (Gandy et al., “Attenuation of Streptozotocin Diabetes with Superoxide Dismutase-Like Copper(II)(3,5-diisopropylsalicylate)2 in the Rat,” Diabetologia 24:437-440 (1983)) and restored the suppressed foxa2 expression in the SKO islets (Wang et al., “Knockouts of SOD1 and GPX1 Exert Different Impacts on Murine Islet Function and Pancreatic Integrity,” Antioxid. Redox Signal. 14:391-401 (2011)). As an essential micronutrient, Se is a component of 25 human selenoproteins (Kryukov et al., “Characterization of Mammalian Selenoproteomes,” Science 300:1439-1443 (2003)) that are involved in antioxidant defense (Brigelius-Flohe, R., “Glutathione Peroxidases and Redox-Regulated Transcription Factors, Biol. Chem. 387:1329-1335 (2006); Brigelius-Flohe et al., “Basic Principles and Emerging Concepts in the Redox Control of Transcription Factors,” Antioxid. Redox Signal. 15:2335-2381 (2011)) and regulation of β-cells (Steinbrenner et al., “Localization and Regulation of Pancreatic Selenoprotein P,” J. Mol. Endocrinol. 50:31-42 (2013)). Either overexpression or deficiency of selenoproteins disturbed glucose homeostasis in mice (Labunskyy et al., “Both Maximal Expression of Selenoproteins and Selenoprotein Deficiency Can Promote Development of Type 2 Diabetes-Like Phenotype in Mice,” Antioxid. Redox Signal. 14:2327-2336 (2011)). It has been reported that Se functioned as an insulin mimetic in isolated adipocytes (Ezaki, O., “The Insulin-Like Effects of Selenate in Rat Adipocytes,” J. Biol. Chem. 265:1124-1128 (1990)) and STZ-induced diabetic rodents (Becker et al., “Oral Selenate Improves Glucose Homeostasis and Partly Reverses Abnormal Expression of Liver Glycolytic and Gluconeogenic Enzymes in Diabetic Rats,” Diabetologia 39:3-11 (1996)). Despite all these well-documented features, little is known of roles and mechanisms of ebselen, CuDIPs, and Se in regulating GSIS.

The present invention is directed to overcoming these and other deficiencies in the art.

### SUMMARY OF THE INVENTION

One aspect of the present invention relates to a method of enhancing glucose-stimulated insulin secretion in a subject. This method involves selecting a subject with: (1) an antioxidant deficiency and (2) a need for enhanced glucose-stimulated insulin secretion, and administering to the selected subject an agent selected from the group consisting of (1) a compound according to Formula I or a pharmaceutically acceptable salt thereof:

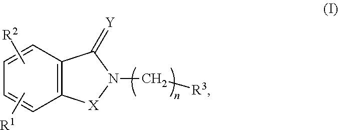

wherein

R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;

R³ is aryl optionally substituted with R⁴;

R⁴ is selected from the group consisting of H, halogen, —OH, —CF₃, —NO₂, —NR⁵R⁶, C₁-C₆ alkyl, and C₁-C₆ alkoxyl;

R⁵ and R⁶ are independently selected from the group consisting of H and C₁-C₆ alkyl;

X is Se or S;

Y is O or S; and

n is 0 to 5, (2) glutathione peroxidase, or (3) activators of PGC-1α antioxidant response element, under conditions effective to enhance glucose-stimulated insulin secretion in the subject.

Another aspect of the present invention relates to a method of treating a subject with Type 2 diabetes. This method involves selecting a subject with: (1) an antioxidant deficiency and (2) Type 2 diabetes, and administering to the selected subject an agent selected from the group consisting of (1) a compound according to Formula I or a pharmaceutically acceptable salt thereof:

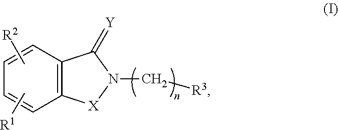

wherein

R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;

R³ is aryl optionally substituted with R⁴;

R⁴ is selected from the group consisting of H, halogen, —OH, —CF₃, —NO₂, —NR⁵R⁶, C₁-C₆ alkyl, and C₁-C₆ alkoxyl;

R⁵ and R⁶ are independently selected from the group consisting of H and C₁-C₆ alkyl;

X is Se or S;

Y is O or S; and

n is 0 to 5, (2) glutathione peroxidase, or (3) activators of PGC-1α antioxidant response element, under conditions effective to treat Type 2 diabetes in the subject.

Another aspect of the present invention relates to a method of treating a subject with hypoglycemia. This method involves selecting a subject with: (1) an antioxidant deficiency and (2) hypoglycemia, and administering to the selected subject an agent selected from the group consisting of (1) a compound according to Formula I or a pharmaceutically acceptable salt thereof:

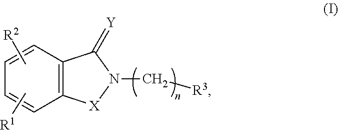

wherein

R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;

R³ is aryl optionally substituted with R⁴;

R⁴ is selected from the group consisting of H, halogen, —OH, —CF₃, —NO₂, —NR⁵R⁶, C₁-C₆ alkyl, and C₁-C₆ alkoxyl;

R⁵ and R⁶ are independently selected from the group consisting of H and C₁-C₆ alkyl;

X is Se or S;

Y is O or S; and

n is 0 to 5, (2) glutathione peroxidase, or (3) activators of PGC-1α antioxidant response element, under conditions effective to treat the subject with hypoglycemia.

Glutathione peroxidase (“GPX”) mimic ebselen and superoxide dismutase (“SOD”) mimic copper diisopropylsalicylate (“CuDIPs”) were used to rescue impaired glucose-stimulated insulin secretion (“GSIS”) in islets of GPX1 and(or) SOD1-knockout mice. Ebselen improved GSIS in islets of all four tested genotypes. The rescue in the GPX1 knockout resulted from a coordinated transcriptional regulation of four key GSIS regulators and was mediated by the peroxisome proliferator-activated receptor γ co-activator 1α (“PGC-1α”)-mediated signaling pathways. In contrast, CuDIPs improved GSIS only in the SOD1 knockout and suppressed gene expression of the PGC-1α pathway.

Islets from the GPX1 and(or) SOD1 knockout mice provided metabolically-controlled intracellular hydrogen peroxide and superoxide conditions for the present study to avoid confounding effects. Bioinformatics analyses of gene promoters and expression profiles guided the search for upstream signaling pathways to link the ebselen-initiated H₂O₂ scavenging to downstream key events of GSIS. The RNA interference was applied to prove PGC-1α as the main mediator for that link.

The experiments described herein revealed a novel metabolic use and clinical potential of ebselen in rescuing GSIS in the GPX1 deficient islets and mice, along with distinct differences between the GPX and SOD mimics in this regard. These findings highlight necessities and opportunities of discretional applications of various antioxidant enzyme mimics in treating insulin secretion disorders.

Knockout of antioxidant enzymes GPX1 and SOD1 alone or together impaired GSIS, but the molecular mechanism and signaling pathway remain unclear. Using islets isolated from the GPX1 and (or) SOD1 knockout mice, applicant has demonstrated that the GPX mimic ebselen rescued impaired GSIS in the GPX1 knockout islets and mice via regulating GK, GLUT2, PDX1, and UCP2 by activating PGC-1α-mediated ARE/GR signaling pathways. In contrast, the SOD mimic CuDIPs showed different roles and mechanisms in regulating GSIS. Applicants' results revealed a novel metabolic effect of ebselen in promoting GSIS and provided a new strategy to treat disorders related to insulin secretion.

Many types of antioxidant enzyme polymorphism (genetic variants in humans have been reported. Some of these alterations are associated with impaired insulin secretion and diabetes. As mentioned above, a Gpx1 gene variant (C198T) lowering the enzyme activity was identified in the South Indian population, which resulted in increased incidences of type 2 diabetes (C/T, 1.4-fold and T/T, 1.8-fold) (Ramprasath et al., “Genetic Association of Glutathione Peroxidase-1 (GPx-1) and NAD(P)H:Quinone Oxidoreductase 1 (NQ01) Variants and Their Association of CAD in Patients with Type-2 Diabetes,” Mol. Cell. Biochem. 361:143-150 (2012), which is hereby incorporated by reference in its entirety). Thus, administration of antioxidant compounds like ebselen or up-regulating their body antioxidant defense may help improve their insulin secretion and function.

### BRIEF DESCRIPTION OF THE DRAWINGS

FIGS. 1A-D are bar graphs showing results that relate to effects of ebselen, CuDIPs, and sodium selenite on GSIS of islets isolated from WT (FIG. 1A), GKO (FIG. 1B), SKO (FIG. 1C), and dKO (FIG. 1D) mice. The islets were pre-treated with ebselen (50 μM in DMSO), CuDIPs (10 μM in ethanol), and sodium selenite (100 nM in saline) or the respective solvent controls for 5 hours. The 16.7 mM glucose stimulation increased insulin secretion (p<0.05 versus 2.8 mM glucose) within all of the treatment groups. **p<0.01 versus DMSO solvent control; ††p<0.01 versus ethanol solvent control; ‡p<0.05, ‡‡p<0.01 versus saline control. Values are means±SEM (n=3).

FIGS. 2A-D are bar graphs showing results that relate to effects of ebselen, CuDIPs, and sodium selenite on GK, GLUT2, PDX1, and UCP2 in islets isolated from WT (FIG. 2A), GKO (FIG. 2B), SKO (FIG. 2C), and dKO (FIG. 2D) mice. GK is shown to have enzyme activity (mU/mg protein), and GLUT2, PDX1, and UCP2 is shown to have relative protein expression by integrated optical densitometry (IOD). *p<0.05, **p<0.01 versus respective solvent control. Values are means±SEM (n=5).

FIGS. 3A-E are bar graphs showing results that relate to effects of the GPX mimic ebselen on GKO islet mRNA levels of gk1, glut2, pdx1, ucp2, and ins1 (FIG. 3A), genes in the ARE pathway (FIG. 3B), genes in the GR pathway (FIG. 3C), genes in the Wnt pathway (FIG. 3D), and genes in the NFAT pathway (FIG. 3E). *p<0.05, **p<0.01 versus control. Values are means±SEM (n=6).

FIGS. 4A-D are bar graphs showing comparison of the ebselen and CuDIPs treatments on the dKO islet mRNA levels of genes in the ARE pathway (FIG. 4A), GR pathway (FIG. 4B), Wnt pathway (FIG. 4C), and NFAT pathway (FIG. 4D). Data are presented as denary logarithm values (stimulation and inhibition are shown as positive and negative values, respectively). *p<0.05, **p<0.01 ebselen versus CuDIPs group. Values are means±SEM (n=6).

FIGS. 5A-C are bar graphs showing results that relate to impacts of knock down (siRNA) of pgc-1α and(or) cebpb on the GKO islet mRNA levels of both genes (FIG. 5A), the ebselen-mediated mRNA changes of gk, glut2, pdx1, and ucp2 (FIG. 5B), and the ebselen-mediated GSIS rescue (FIG. 5C). *p<0.05, **p<0.01 versus control in panels A and B, and means with different letters a, b, and c, p<0.05 in panel C. Values are means±SEM (n=6).

FIGS. 6A-B are graphs showing results that relate to in vivo effects of ebselen on the GKO mouse GSIS (FIG. 6A) and GTT (FIG. 6B). The ebselen (50 mg/kg) was injected (i.p.) into fasting (overnight for 8 h) GKO mice at 1 hour before the glucose challenge (1 g/kg) and DMSO was injected as the solvent control. GTT data are presented as relative values (fasting blood glucose before ebselen injection was defined as 100). *p<0.05 versus control. Values are means±SEM (n=6).

FIGS. 7A-D show effects of ebselen, CuDIPs, and sodium selenite on GLUT2, PDX1, and UCP2 protein levels in islets isolated from WT (FIG. 7A), GKO (FIG. 7B), SKO (FIG. 7C), and dKO (FIG. 7D) mice. The blots are representatives of five independent Western-blot analyses. + represents treated and − represents respective solvent control groups.

FIGS. 8A-C are bar graphs showing effects of ebselen on the WT and GKO islet H₂O₂ levels (FIG. 8A), LDH activity (FIG. 8B), and GSR activity (FIG. 8C). Values are means±SEM (n=3). Means with different letters a, b, and c are different (p<0.01) in panel A.

FIG. 9 is an illustration showing the bioinformatics-derived signaling pathways for expressions of GK, GLUT2, PDX1, and UCP2 regulated by ebselen. The dotted boxes indicate four different signaling pathways: (A) ARE, (B) GR, (C) Wnt, and (D) NFAT.

FIGS. 10A-D are bar graphs showing effects of the ebselen injection to the GKO mice on activities of plasma ALT (FIG. 10A), plasma AKP (FIG. 10B), liver LDH (FIG. 10C), and liver GSR (FIG. 10D). The ebselen (50 mg/kg) was injected (i.p.) into the fasting (overnight for 8 h) GKO mice at 1 hour before tissue collection. The same volume of DMSO was injected as the solvent control. Values are means±SEM (n=5).

### DETAILED DESCRIPTION OF THE INVENTION

One aspect of the present invention relates to a method of enhancing glucose-stimulated insulin secretion in a subject. This method involves selecting a subject with: (1) an antioxidant deficiency and (2) a need for enhanced glucose-stimulated insulin secretion, and administering to the selected subject an agent selected from the group consisting of (1) a compound according to Formula I or a pharmaceutically acceptable salt thereof:

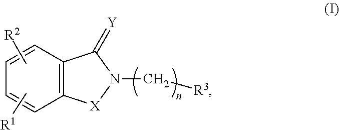

wherein

R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;

R³ is aryl optionally substituted with R⁴;

R⁴ is selected from the group consisting of H, halogen, —OH, —CF₃, —NO₂, —NR⁵R⁶, C₁-C₆ alkyl, and C₁-C₆ alkoxyl;

R⁵ and R⁶ are independently selected from the group consisting of H and C₁-C₆ alkyl;

X is Se or S;

Y is O or S; and

n is 0 to 5, (2) glutathione peroxidase, or (3) activators of PGC-1α antioxidant response element, under conditions effective to enhance glucose-stimulated insulin secretion in the subject.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “halogen” means fluoro, chloro, bromo, or iodo.

The term “alkyl” means an aliphatic hydrocarbon group which may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “alkoxy” or “alkoxyl” means groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy, and the like. Lower-alkoxy refers to groups containing one to four carbons. For the purposes of the present patent application, alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

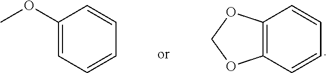

The term “aryl” means an aromatic monocyclic or multicyclic ring system of 6 to about 14 carbon atoms, preferably of 6 to about 10 carbon atoms. Representative aryl groups include phenyl and naphthyl.

The term “monocyclic” used herein indicates a molecular structure having one ring.

The term “polycyclic” or “multicyclic” used herein indicates a molecular structure having two or more rings, including, but not limited to, fused, bridged, or spiro rings.

The term “compounds of the invention”, and equivalent expressions, are meant to embrace compounds of general formula (I) as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The term “pharmaceutically acceptable salts” means the relatively non-toxic, inorganic, and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418, which are hereby incorporated by reference in their entirety). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include, for example, sodium hydride, sodium hydroxide, potassium hydroxide, calcium hydroxide, aluminium hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “activators of PGC-1α antioxidant response element” refers to compounds that activate PGC-1α-mediated ARE/GR signaling pathway.

In another embodiment, in the compound of Formula I, n is 0, R¹ is H, R² is H, R³ is phenyl, X is Se, and Y is O.

While it may be possible for compounds of Formula (I) to be administered as raw chemicals, it will often be preferable to present them as a part of a pharmaceutical composition. Accordingly, another aspect of the present invention is a pharmaceutical composition containing a therapeutically effective amount of the compound of Formula (I), or a pharmaceutically acceptable salt or solvate thereof, and a pharmaceutically acceptable carrier. The carrier must be “acceptable” in the sense of being compatible with the other ingredients of the formulation and not deleterious to the recipient thereof.

The term “therapeutically effective amounts” is meant to describe an amount of compound of the present invention effective in increasing the levels of serotonin, norepinephrine, or dopamine at the synapse and thus producing the desired therapeutic effect. Such amounts generally vary according to a number of factors well within the purview of ordinarily skilled artisans given the description provided herein to determine and account for. These include, without limitation: the particular subject, as well as its age, weight, height, general physical condition, and medical history; the particular compound used, as well as the carrier in which it is formulated and the route of administration selected for it; and, the nature and severity of the condition being treated.

The term “pharmaceutical composition” means a composition comprising a compound of Formula (I), glutathione peroxidase, or activators of PGC-1α antioxidant response element and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgement, suitable for use in contact with the cells of humans and lower animals without undue toxicity, irritation, allergic response and the like, and are commensurate with a reasonable benefit/risk ratio.

The term “pharmaceutically acceptable dosage forms” means dosage forms of the compound of the invention, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition.

In practicing the method of the present invention, agents suitable for treating a subject can be administered using any method standard in the art. The agents, in their appropriate delivery form, can be administered orally, intradermally, intramuscularly, intraperitoneally, intravenously, subcutaneously, or intranasally. The compositions of the present invention may be administered alone or with suitable pharmaceutical carriers, and can be in solid or liquid form, such as tablets, capsules, powders, solutions, suspensions, or emulsions. In one embodiment, administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

The agents of the present invention may be orally administered, for example, with an inert diluent, or with an assimilable edible carrier, or it may be enclosed in hard or soft shell capsules, or it may be compressed into tablets, or they may be incorporated directly with the food of the diet. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. (Abuchowski and Davis, “Soluble Polymer-Enzyme Adducts,” In: Enzymes as Drugs, Hocenberg and Roberts, eds., Wiley-Interscience, New York, N.Y., pp. 367-383 (1981), which are hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agents of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The agents of the present invention may also be administered directly to the airways in the form of an aerosol. For use as aerosols, the agent of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer.

The percentage of active ingredient in the compositions of the present invention may be varied, it being necessary that it should constitute a proportion such that a suitable dosage shall be obtained. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.01 to about 100 mg/kg body weight, preferably about 0.01 to about 10 mg/kg body weight per day by inhalation, from about 0.01 to about 100 mg/kg body weight, preferably 0.1 to 70 mg/kg body weight, more especially 0.1 to 10 mg/kg body weight per day by oral administration, and from about 0.01 to about 50 mg/kg body weight, preferably 0.01 to 10 mg/kg body weight per day by intravenous administration. In each particular case, the doses will be determined in accordance with the factors distinctive to the subject to be treated, such as age, weight, general state of health, and other characteristics which can influence the efficacy of the medicinal product.

The products according to the present invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. It goes without saying that, for other patients, it will be necessary to prescribe not more than one or two doses per day.

Another aspect of the present invention relates to a method of treating a subject with Type 2 diabetes. This method involves selecting a subject with: (1) an antioxidant deficiency and (2) Type 2 diabetes, and administering to the selected subject an agent selected from the group consisting of (1) a compound according to Formula I or a pharmaceutically acceptable salt thereof:

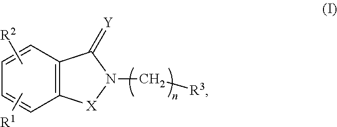

wherein

R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;

R³ is aryl optionally substituted with R⁴;

R⁴ is selected from the group consisting of H, halogen, —OH, —CF₃, —NO₂, —NR⁵R⁶, C₁-C₆ alkyl, and C₁-C₆ alkoxyl;

R⁵ and R⁶ are independently selected from the group consisting of H and C₁-C₆ alkyl;

X is Se or S;

Y is O or S; and

n is 0 to 5, (2) glutathione peroxidase, or (3) activators of PGC-1α antioxidant response element, under conditions effective to treat Type 2 diabetes in the subject.

This method is carried out using the formulations and modes of administration described above.

The term “method of treating” means amelioration or relief from the symptoms and/or effects associated with the disorders described herein. As used herein, reference to “treatment” of a patient is intended to include prophylaxis.

Another aspect of the present invention relates to a method of treating a subject with hypoglycemia. This method involves selecting a subject with: (1) an antioxidant deficiency and (2) hypoglycemia, and administering to the selected subject an agent selected from the group consisting of (1) a compound according to Formula I or a pharmaceutically acceptable salt thereof:

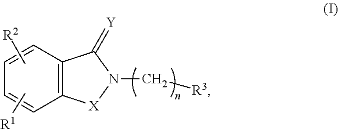

wherein

R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;

R³ is aryl optionally substituted with R⁴;

R⁴ is selected from the group consisting of H, halogen, —OH, —CF₃, —NO₂, —NR⁵R⁶, C₁-C₆ alkyl, and C₁-C₆ alkoxyl;

R⁵ and R⁶ are independently selected from the group consisting of H and C₁-C₆ alkyl;

X is Se or S;

Y is O or S; and

n is 0 to 5, (2) glutathione peroxidase, or (3) activators of PGC-1α antioxidant response element, under conditions effective to treat the subject with hypoglycemia.

This method is carried out using the formulations and modes of administration described above.

### EXAMPLES

### Example 1

### Mouse Models, Animal Care, and In Vivo Study

Mouse experiments were approved by the Institutional Animal Care and Use Committee at Cornell University and conducted in accordance with National Institutes of Health guidelines for animal care. The GPX1 knockout (“GKO”), SOD1 knockout (“SKO”), and their double knockout (“dKO”) mice were derived from the C57BL/6 line. Deletions of gpx1 and sod1 genes in respective genotypes were verified by PCR analysis using tail DNA as templates and enzyme activity assays of various tissues (Lei et al., “Mice Deficient in Cu,Zn-Superoxide Dismutase are Resistant to Acetaminophen Toxicity,” Biochem. J. 399:455-461 (2006), which is hereby incorporated by reference in its entirety). All experimental mice were 3 to 6 month-old males, weaned at 3 weeks of age, reared in plastic cages in an animal room at a constant temperature (22° C.) with a 12 hour light-dark cycle, and were given free access to a Torula yeast and sucrose based diet added with 0.3 mg Se/kg (as sodium selenite) (Wang et al., “Knockouts of SOD1 and GPX1 Exert Different Impacts on Murine Islet Function and Pancreatic Integrity,” Antioxid. Redox Signal. 14:391-401 (2011), which is hereby incorporated by reference in its entirety), and distilled water.

To examine in vivo effect of ebselen on insulin secretion, fasting (overnight for 8 hours), GKO mice (3 month-old male, n=6 per group) were given an i.p. injection of ebselen (at 50 mg/kg body weight) at 1 hour prior to the glucose tolerance test (GTT, 1 g of glucose/kg) and glucose-stimulated insulin secretion (“GSIS”) test (McClung et al., “Development of Insulin Resistance and Obesity in Mice Overexpressing Cellular Glutathione Peroxidase,” Proc. Natl. Acad. Sci. USA 101:8852-8857 (2004), which is hereby incorporated by reference in its entirety). Ebselen was dissolved in dimethyl sulfoxide (DMSO) and injected with saline (1:1 by volume) together. The same volume of DMSO and saline was used as the solvent control. Blood glucose concentrations were measured by clipping tails and using the Glucometer Elite system (Bayer, Elkhart, Ind.). Plasma insulin concentrations were determined using a rat/mouse Insulin ELISA kit with mouse insulin as standards (Crystal Chem, Downers Grove, Ill.).

### Example 2

### In Vitro Experiments

All chemicals were purchased from Sigma (Saint Louis, Mo.) unless indicated otherwise. Detailed protocols, reagents, and instruments for islet isolation, culture, and insulin secretion were the same as described previously (Wang et al., “Molecular Mechanisms for Hyperinsulinaemia Induced by Overproduction of Selenium-Dependent Glutathione Peroxidase-1 in Mice,” Diabetologia 51:1515-1524 (2008), which is hereby incorporated by reference in its entirety). Briefly, Langerhans' islets were isolated from the WT, GKO, SKO, and dKO mice using a standard procedure (Gotoh et al., “An Improved Method for Isolation of Mouse Pancreatic Islets,” Transplantation 40:437-438 (1985), which is hereby incorporated by reference in its entirety) with minor modifications. Isolated islets (50 per sample, n=3 per group) were recovered in RPMI 1640 (Gibco, Grand Island, N.Y.) with 5.5 mM glucose and 10% FBS for 2 hours before treatment. For the islet experiments, ebselen, CuDIPs, and Se (sodium selenite) were used at 50, 10, and 0.1 μM and prepared in DMSO, ethanol, and saline, respectively. The same amounts of vehicle were used as the respective controls. The chemicals were removed after 5 hour incubation, and the islets were transferred to Krebs-Henseleit buffer for 30 min and then incubated for 60 min with 2.8 or 16.7 mM glucose in the same buffer. The supernatants were collected for insulin analysis and the islets were used for mRNA, protein, and enzyme activity analyses.

### Example 3

### Real Time Q-PCR and Western Blot Analyses

Total RNA was extracted from islets using Trizol reagent (Invitrogen, Carlsbad, Calif.). Reverse transcription was performed using Super Script III reverse transcriptase, RNaseOUT Ribonuclease Inhibitor, and Oligo(dT)₁₂₋₁₈ (Invitrogen). The cDNA obtained from 1 μg of total RNA was used as a template for PCR amplification. Oligonucleotide primers were designed based on Genebank entries and IDT PrimerQuest. Primer sequences for the GSIS-related genes are described in Table 1 below. Relative mRNA levels were determined by real time Q-PCR (7900HT; Applied Biosystems, Foster City, Calif.) as previously described (Pepper et al., “Impacts of Dietary Selenium Deficiency on Metabolic Phenotypes of Diet-Restricted GPX1-Overexpressing Mice,” Antioxid. Redox Signal. 14:383-390 (2011), which is hereby incorporated by reference in its entirety).

TABLE 1
Primers list for Real time Q-PCR

| | | | | Tm (For, | |
|---|---|---|---|---|---|
| | gene | Forward Primer | Reverse Primer | Rev) | Size |
| | HPRT | TTTCCCTGGTTAAGCAGTACAGCCC | TGGCCTGTATCCAACACTTCGAGA | 60.6, 59.6 | 89 |
| | | (SEQ ID NO: 1) | (SEQ ID NO: 2) | | |
| ARE pathway | CAMK-II | TGAGGACCAACACAAGCTGTACCA | TGGTCAGCATCTGGTTGATGAGGT | 60.0, 60.0 | 116 |
| | | (SEQ ID NO: 3) | (SEQ ID NO: 4) | | |
| | CREB1 | ACAGCAGATTCTAGTGCCCAGCAA | TCTTCAGCAGGCTGTGTAGGAAGT | 60.4, 59.6 | 153 |
| | | (SEQ ID NO: 5) | (SEQ ID NO: 6) | | |
| | SIRT1 | CCTTTCAGAACCACCAAAGCGGAA | AAGTCAGGAATCCCACAGGAGACA | 59.6, 59.3 | 138 |
| | | (SEQ ID NO: 7) | (SEQ ID NO: 8) | | |
| | PGC1a | AGCACTCAGAACCATGCAGCAAAC | TTTGGTGTGAGGAGGGTCATCGTT | 60.0, 60.1 | 183 |
| | | (SEQ ID NO: 9) | (SEQ ID NO: 10) | | |
| | Nrf1 | AGTGATGTCCGCACAGAAGAGCAA | GTGGCCTGAGTTTGTGTTTGCTGA | 60.4, 59.9 | 143 |
| | | (SEQ ID NO: 11) | (SEQ ID NO: 12) | | |
| | Nrf2 | AGGTTGCCCACATTCCCAAACAAG | TGAAGACTGAACTTTCAGCGTGGC | 60.0, 59.4 | 138 |
| | | (SEQ ID NO: 13) | (SEQ ID NO: 14) | | |
| | FoxO1 | AAGAGCGTGCCCTACTTCAAGGAT | GTGAAGGGACAGATTGTGGCGAAT | 59.9, 59.2 | 87 |
| | | (SEQ ID NO: 15) | (SEQ ID NO: 16) | | |
| | HNF4a | TTCGGCATGGCCAAGATTGACAAC | TTGGTGCCCATGTGTTCTTGCATC | 60.1, 60.1 | 122 |
| | | (SEQ ID NO: 17) | (SEQ ID NO: 18) | | |
| | PPARg | ACATAAAGTCCTTCCCGCTGACCA | AAATTCGGATGGCCACCTCTTTGC | 59.9, 60.0 | 180 |
| | | (SEQ ID NO: 19) | (SEQ ID NO: 20) | | |
| GR pathway | GR | GCAGTGAAATGGGCAAAGGCGATA | CCAGGGCAAATGCCATGAGAAACA | 59.9, 60.0 | 106 |
| | | (SEQ ID NO: 21) | (SEQ ID NO: 22) | | |
| | SMAD4 | TGTCCACAGGACAGAAGCGATTGA | ATCTTATGAACAGCGTCGCCAGGT | 59.9, 60.2 | 180 |
| | | (SEQ ID NO: 23) | (SEQ ID NO: 24) | | |
| | Cebpb | ACAAGCTGAGCGACGAGTACAAGA | GACAGCTGCTCCACCTTCTTCTG | 59.7, 59.5 | 160 |
| | | (SEQ ID NO: 25) | (SEQ ID NO: 26) | | |
| | Nr2f1 | TTCAGGAACAGGTGGAGAAGCTCA | TTTCTCCTGCAGGCTTTCGATGTG | 59.6, 59.4 | 137 |
| | | (SEQ ID NO: 27) | (SEQ ID NO: 28) | | |
| | Nr2f2 | TCCAAGAGCAAGTGGAGAAGCTCA | ACTCTTCCAAAGCACACTGGGACT | 59.8, 60.1 | 159 |
| | | (SEQ ID NO: 29) | (SEQ ID NO: 30) | | |
| | PXR | TGATGGACGCTCAGATGCAAACCT | AGAAACTCTGGAAGCTCACAGCCA | 60.5, 60.0 | 103 |
| | | (SEQ ID NO: 31) | (SEQ ID NO: 32) | | |
| | RXRa | TGACATGCAGATGGACAAGACGGA | TGCAGTACGCTTCTAGTGACGCAT | 60.0, 60.0 | 140 |
| | | (SEQ ID NO: 33) | (SEQ ID NO: 34) | | |
| Wnt pathway | Akt1 | AGGCCGCTACTATGCCATGAAGAT | TGGAATGAGTACTTGAGGGCCGTA | 59.9, 59.2 | 138 |
| | | (SEQ ID NO: 35) | (SEQ ID NO: 36) | | |
| | Gsk3b | TTGGAGCCACTGATTACACGTCCA | TTCCACCAACTGATCCACACCACT | 60.0, 60.1 | 119 |
| | | (SEQ ID NO: 37) | (SEQ ID NO: 38) | | |
| | Ctnnb1 | TGGGACTCTGCACAACCTTTCTCA | AGTGTCGTGATGGCGTAGAACAGT | 60.0, 59.8 | 132 |
| | | (SEQ ID NO: 39) | (SEQ ID NO: 40) | | |
| | Tcf7l2 | AGAGAGTGCAGCCATCAACCAGAT | CTGCATGTGAAGCTGTCGTTCCTT | 60.0, 59.5 | 112 |
| | | (SEQ ID NO: 41) | (SEQ ID NO: 42) | | |
| | Tcf7 | TGAGAGCCAAGGTCATTGCTGAGT | TTCCTTGCGGGCCAGTTCATAGTA | 60.1, 59.9 | 128 |
| | | (SEQ ID NO: 43) | (SEQ ID NO: 44) | | |
| | Cdx1 | AGGAGTTTCACTACAGCCGGTACA | CTGCTGCTGCTGCTGTTTCTTCTT | 59.3, 59.9 | 152 |
| | | (SEQ ID NO: 45) | (SEQ ID NO: 46) | | |
| NFAT pathway | Nfatc4 | ATGGTGGCTACAGCCAGCTATGAA | TCACCCTTCCGTAGCTCAATGTCT | 60.1, 59.4 | 149 |
| | | (SEQ ID NO: 47) | (SEQ ID NO: 48) | | |
| | CREBBP | ATGCCCAATGTTTCCAACGACCTG | GCCCAGCATGCAGATGAATCACAA | 59.9, 60.0 | 94 |
| | | (SEQ ID NO: 49) | (SEQ ID NO: 50) | | |
| | Ppp3ca | TGTGTACACGGTGGTTTGTCTCCA | ACAGCCTCTGACTGTGTTGTGAGT | 60.1, 59.9 | 180 |
| | | (SEQ ID NO: 51) | (SEQ ID NO: 52) | | |
| | Nkx2.5 | AAGTGCTCTCCTGCTTTCCCA | TTTGTCCAGCTCCACTGCCTTCT | 58.5, 60.5 | 132 |
| | | (SEQ ID NO: 53) | (SEQ ID NO: 54) | | |
| | E12 | TTCCTTTGACCCTAGCCGGACATA | AACACTGGTGTCTCTCCCAAAGGT | 59.3, 59.9 | 151 |
| | | (SEQ ID NO: 55) | (SEQ ID NO: 56) | | |
| | GATA4 | AGGGTGAGCCTGTATGTAATGCCT | AGGACCTGCTGGCGTCTTAGATTT | 59.7, 59.9 | 143 |
| | | (SEQ ID NO: 57) | (SEQ ID NO: 58) | | |
| GSIS related genes | GK | AGGGAACAACATCGTGGGACTTCT | GTGTCATTCACCATTGCCACCACA | 59.9, 59.9 | 87 |
| | | (SEQ ID NO: 59) | (SEQ ID NO: 60) | | |
| | GLUT2 | AAAGGAAGAGGCATCGACTGAGCA | ACACCAATGGTTGCATACACAGGC | 60.1, 59.9 | 198 |
| | | (SEQ ID NO: 61) | (SEQ ID NO: 62) | | |
| | PDX1 | AGCTCCCTTTCCCGTGGATGAAAT | TAGGCAGTACGGGTCCTCTTGTTT | 60.3, 59.4 | 112 |
| | | (SEQ ID NO: 63) | (SEQ ID NO: 64) | | |
| | UCP2 | AGCCTACAAGACCATTGCACGAGA | ATAGGTCACCAGCTCAGCACAGTT | 60.0, 59.9 | 109 |
| | | (SEQ ID NO: 65) | (SEQ ID NO: 66) | | |
| | INS1 | TAAAGCTGGTGGGCATCCAGTAAC | GGGACCACAAAGATGCTGTTTGAC | 58.9, 58.3 | 174 |
| | | (SEQ ID NO: 67) | (SEQ ID NO: 68) | | |

Islet samples used for Western blot analysis were homogenized in phosphate buffer (50 mM, pH 7.4) containing 0.1% Triton X-100 and protease inhibitor mixture (AEBSF, aprotinin, bestatin hydrochloride, E-64-[N-(transepoxysuccinyl)-L-leucine 4-guanidinobutylamide], leupeptin, and pepstatin A). The homogenates were centrifuged at 14000×g for 10 min at 4° C. A total of 10 μg of protein per lane was subjected to Western blot analysis (McClung et al., “Development of Insulin Resistance and Obesity in Mice Overexpressing Cellular Glutathione Peroxidase,” Proc. Natl. Acad. Sci. USA 101:8852-8857 (2004), which is hereby incorporated by reference in its entirety). After the gel electrophoresis, the separated proteins were transferred onto a protran BA85 nitrocellulose membrane (Schleicher Schuell Bioscience, Keene, N.H.). The membranes were incubated first with respective primary antibodies (rabbit anti-GLUT2, anti-PDX1, and anti-UCP2, Millipore, Billerica, Mass.), and thereafter the second antibody against rabbit IgG (Bio-Rad, Hercules, Calif.). For loading and transfer normalization, the rabbit anti-β-actin antibody (Cell Signaling, Beverly, Mass.) was used. An enhanced chemiluminescent kit (Pierce, Rockford, Ill.) was used for detection of the band intensity.

### Example 4

### Enzyme Activity Measures

Islet GK activity was assayed at 28° C. by measuring absorbance increases of NADPH at 340 nm in a coupled enzyme system. One unit of GK activity was defined as the amount that catalyzes the formation of 1 μmole of glucose 6-phosphate per minute. Islet and liver LDH activities were measured using a kit from Sigma according to the manufacturer's instructions. Islet and liver GSR activities were measured as previously-described (Zhu et al., “Double Null of Selenium-Glutathione Peroxidase-1 and Copper, Zinc-Superoxide Dismutase Enhances Resistance of Mouse Primary Hepatocytes to Acetaminophen Toxicity,” Exp. Biol. Med. (Maywood) 231:545-552 (2006), which is hereby incorporated by reference in its entirety) and one unit of activity was defined as 1 nmol of glutathione disulfide reduced per minute. Plasma ALT activity was assayed using a kit (Thermo Scientific, Waltham, Mass.) according to the manufacturer's instructions. Plasma AKP activity was measured by the hydrolysis of p-nitrophenol phosphate to p-nitrophenol, and the enzyme activity unit was defined as the amount of activity that releases 1 μmol of p-nitrophenol per minute at 30° C. (Bowers et al., “A Continuous Spectrophotometric Method for Measuring the Activity of Serum Alkaline Phosphatase,” Clin. Chem. 12:70-89 (1966), which is hereby incorporated by reference in its entirety).

### Example 5

### Intracellular ROS

Islet ROS levels were determined using the fluorescent probe, 2′,7′-dichlorofluorescin diacetate (“DCFH-DA”). Briefly, the treated islets were washed and incubated with DCFH-DA (20 μM) for 10 min at 37° C. After removal of the probe, cells were washed with pre-warmed PBS. Fluorescence was monitored at 488 nm excitation and 525 nm emission wavelengths.

### Example 6

### RNA Interference

Silencer Selected Pre-Designed siRNAs (Invitrogen) duplex sequences that specifically target pgc-1α [NM_{—}008904.2] and c/ebpβ [NM_{—}009883.3] were employed. The Silencer® Selected Negative Control from Invitrogen was used as non-targeting scramble siRNA. The transfection was performed according to the manufacturer's recommendations with minor modifications. Briefly, islets were dispersed into single cells by mechanical shaking at 37° C. for 3 min in 0.05% trypsin with 0.5 mM EDTA (Ianus et al., “In Vivo Derivation of Glucose-Competent Pancreatic Endocrine Cells from Bone Marrow Without Evidence of Cell Fusion,” J. Clin. Invest. 111:843-850 (2003), which is hereby incorporated by reference in its entirety). The enzymatic reaction was stopped by adding 10% BSA. Cells were washed and recovered in full culture medium at 37° C. for 2 hours. Thereafter, islet cells were divided equally into 24-well plates in culture medium without antibiotics or fetal bovine serum. The siRNAs (20 nM) was transfected twice using Lipofectamine™ RNAiMAX (Invitrogen). The second transfection was performed 24 hours after the first. The efficiency of siRNA was verified by real time Q-PCR analysis of the target genes. After the second transfection, the islets were recovered in RPMI 1640 culture medium with 5.5 mM glucose and 10% fetal bovine serum for 2 hours. Subsequently, the ebselen treatment and the GSIS test were performed as described above. The medium supernatants and the islets were collected for insulin and mRNA analyses, respectively.

### Example 7

### Bioinformatics Analyses of Gene Promoter Sequences

Upstream (1,000 base-pairs) genomic sequences of gk, glut2, pdx1, and ucp2 were retrieved from EMBL database (http://www.ensembl.org/). The transcription factor binding sites were predicted using TESS database (http://www.cbil.upenn.edu/tess) and compared with related factors in beta cells in Gene Interactions Database from BCBC (http://genomics.betacell.org/gbco/home.jsp). The candidate transcription factors were then submitted to the KEGG pathway database, and were sorted into different signaling pathways.

### Example 8

### Statistical Analysis

Data were analyzed using SAS (release 6.11, SAS Institute, Cary, N.C.). Treatment effects were determined by one-way or two-way ANOVA. Results are presented as mean±SEM and significance level was set at p<0.05.

### Example 9

### Results

Effects of Ebselen, CuDIPs, and Se on GSIS of Islets

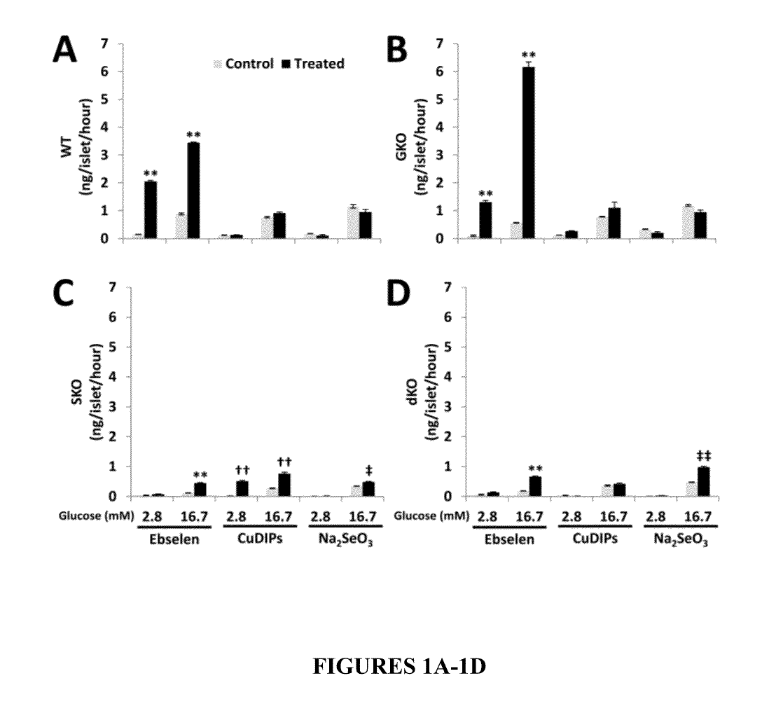

Ebselen, CuDIPs, and Se were used as the GPX mimic, SOD mimic, and compound incorporated as selenocysteine into the active center of GPX, respectively, and incubated with islets isolated from the GKO, SKO and dKO mice, along with the WT, to rescue their impaired GSIS. Ebselen enhanced (p<0.01) GSIS of islets of all four genotypes treated with 16.7 mM glucose (FIGS. 1A-D), compared with their respective controls. Notably, such enhancement was so strong in the GKO islets that their GSIS even exceeded the WT level. The ebselen treatment also elevated (p<0.01) the baseline insulin secretions of WT and GKO islets (at 2.8 mM glucose). In contrast, CuDIPs promoted GSIS and the baseline insulin secretion only in the SKO islets (p<0.01). Meanwhile, Na₂SeO₃ elevated islet GSIS of SKO (p<0.05) and dKO (p<0.01).

Responses of GK, GLUT2, PDX1, UCP2, LDH, GSR, and ROS

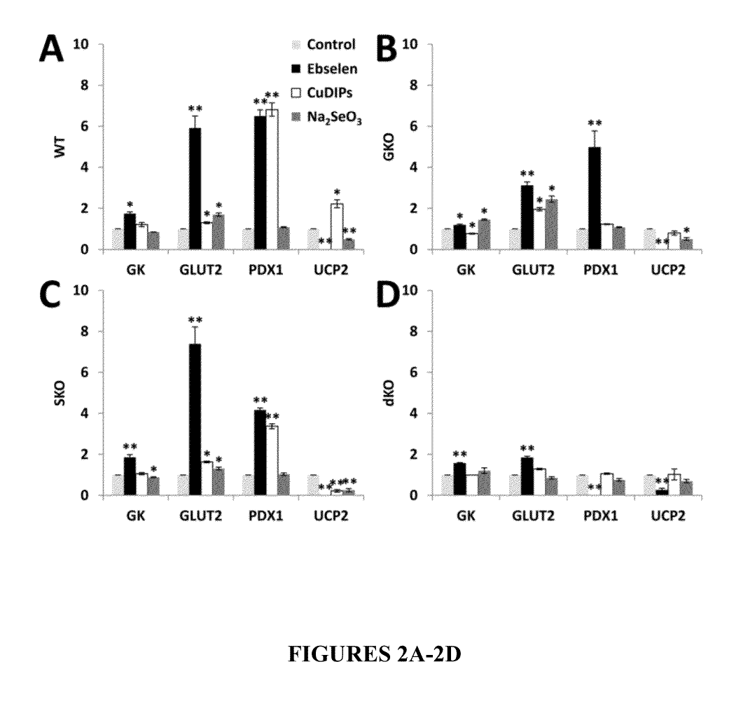

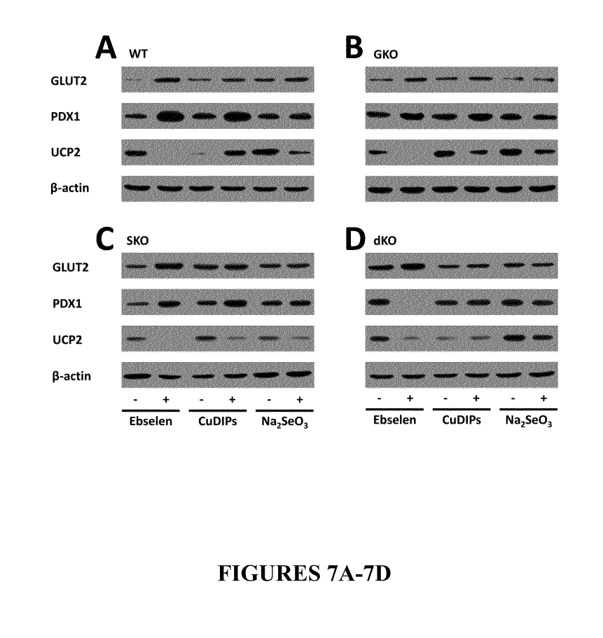

To explore biochemical mechanisms for the above-observed GSIS rescues by ebselen, CuDIPs, and Se, their effects on the activity or protein levels of four key regulators (GK, GLUT2, PDX1, and UCP2) of GSIS were determined. Ebselen elevated (p<0.05) GK activity by 74%, 20%, 86%, and 58% and GLUT2 by 4.9-fold, 2.1-fold, 6.4-fold, and 85%, respectively, in the WT, GKO, SKO, and dKO islets (FIGS. 2A-D, FIGS. 7A-D), compared with their respective controls. Meanwhile, ebselen diminished (p<0.01) islet UCP2 in all genotypes. While ebselen elevated (p<0.01) PDX1 in WT (5.5-fold), GKO (4.0-fold), and SKO (3.2-fold) islets, it actually blocked (p<0.01) the same protein in the dKO islets. In the SKO islets, CuDIPs elevated (p<0.05) GLUT2 and PDX1 by 63% and 2.4-fold, respectively, but decreased (p<0.01) UCP2 by 78%. While CuDIPs increased (p<0.05) GLUT2, PDX1, and UCP2 in the WT islets (FIG. 2A), it caused opposite changes in GK activity (decrease) and GLUT2 (increase) in the GKO islets (FIG. 2B). Compared with the controls, the Na₂SeO₃ treatment increased (p<0.05) GLUT2 by 31% to 1.5-fold, but decreased (p<0.05) UCP2 by 49% to 75%, respectively, in the WT, GKO, and SKO islets. The same treatment increased (p<0.05) GK activity in the GKO islets, but decreased (p<0.05) that in the SKO islets. Neither CuDIPs nor Na₂SeO₃ affected any of these four proteins in the dKO islets (FIG. 2D).

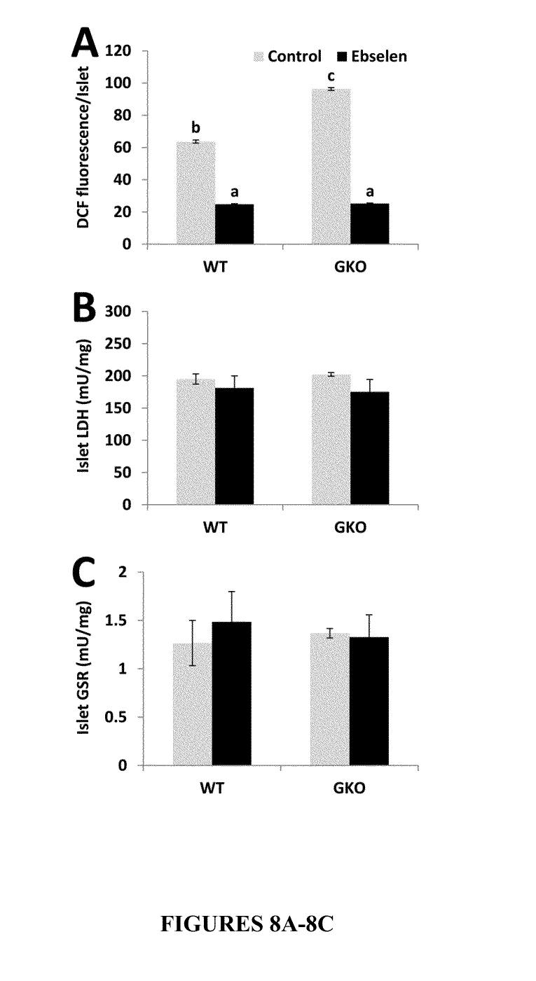

Impacts of the GKO and ebselen on islet intracellular H₂O₂ status were indirectly assessed by a non-specific ROS probe (DCF, FIG. 8A). Ebselen decreased the intracellular ROS by 74% in the GKO islets and removed their initial genotype difference from the WT islets. However, the ebselen treatment showed no effect on activities of two thiol-containing enzymes: lactate dehydrogenase (“LDH”) and glutathione reductase (“GSR”) in the WT and GKO islets (FIGS. 8B-C).

Signaling Mapping for GSIS Regulation by Ebselen

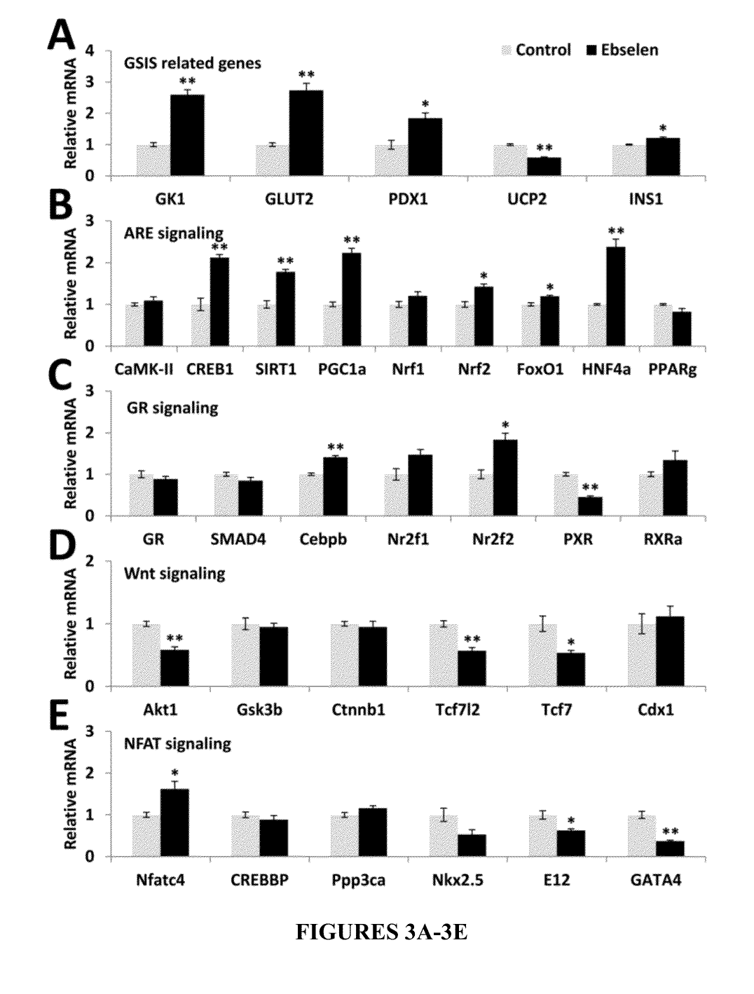

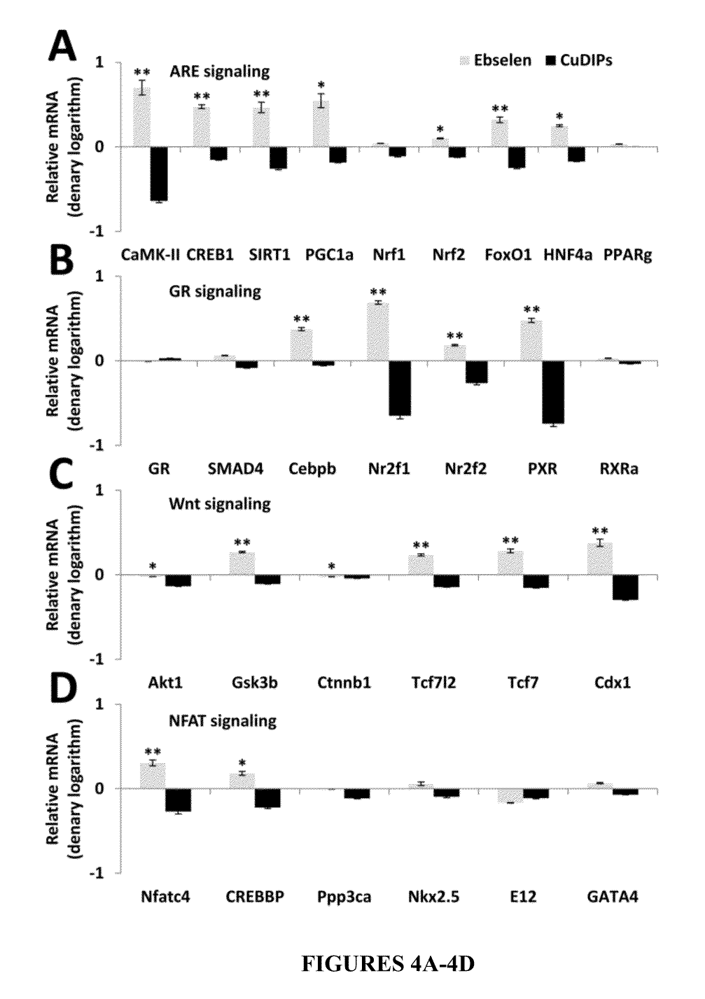

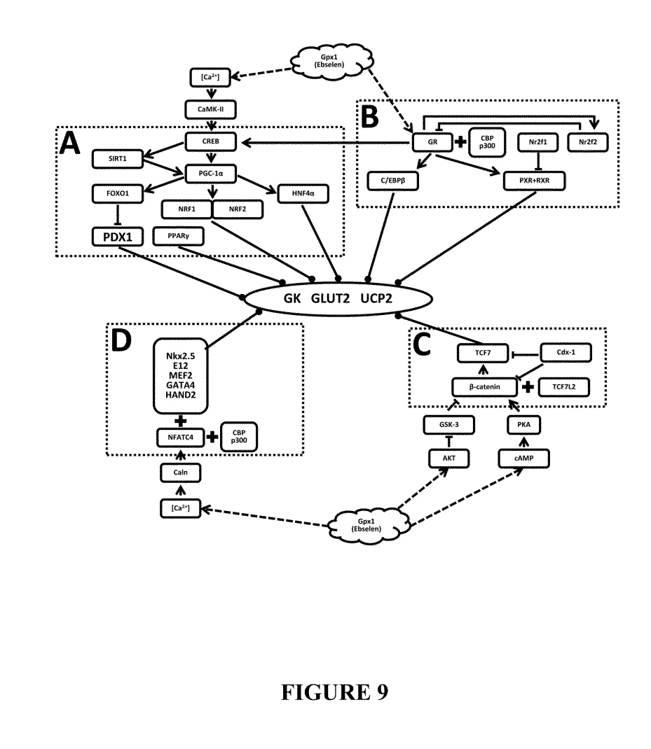

To reveal if the illustrated effects of ebselen on islet GK, GLUT2, PDX1, and UCP2 were mediated by transcriptional regulation, their mRNA responses to ebselen in the GKO islets were determined by Q-PCR. Because their mRNA changes resembled those of their protein responses (FIG. 3A), a transcriptional regulation was proposed for this cascade and their gene promoter regions were analyzed to search for shared binding domains that might mediate the regulation. After 1,000 base-pairs upstream genomic sequences of each gene were retrieved from EMBL database (http://www.ensembl.org/), the sequences were submitted to Transcription Element Search System (TESS) database (http://www.cbil.upenn.edu/tess) for transcriptional factor binding domain prediction. The common binding domains of these four genes with p<0.05 were submitted to Beta Cell Biology Consortium (BCBC) Gene Interactions Databases (http://genomics.betacell.org/gbco/home.jsp) for gene relationship comparison. The predicted transcriptional factor binding domains and their related proteins were submitted to Kyoto Encyclopedia of Genes and Genomes (KEGG) database (http://www.genome.jp/kegg/) for signaling pathway mapping. This bioinformatics approach identified the PGC-1α-mediated ARE and other three signaling pathways (GR, Wnt, and NFAT, FIG. 9). Subsequent Q-PCR analysis showed that 6 out of the 9 genes in the PGC-1α-mediated ARE pathway (FIG. 3B) and 2 out of the 7 genes in the GR pathway (FIG. 3C) were up-regulated by ebselen. Meanwhile, 3 out 6 genes in the Wnt pathway (FIG. 3D) and 2 out of 6 genes in the NFAT pathway (FIG. 3E) were down-regulated by ebselen. After the dKO islets were treated with ebselen, many genes in these four pathways were up-regulated (p<0.05) compared with the controls (FIGS. 4A-D). In contrast, CuDIPs suppressed (p<0.05) expressions of these genes in the dKO islets.

PGC-1α as a Main Mediator for the Upstream Signaling Regulation

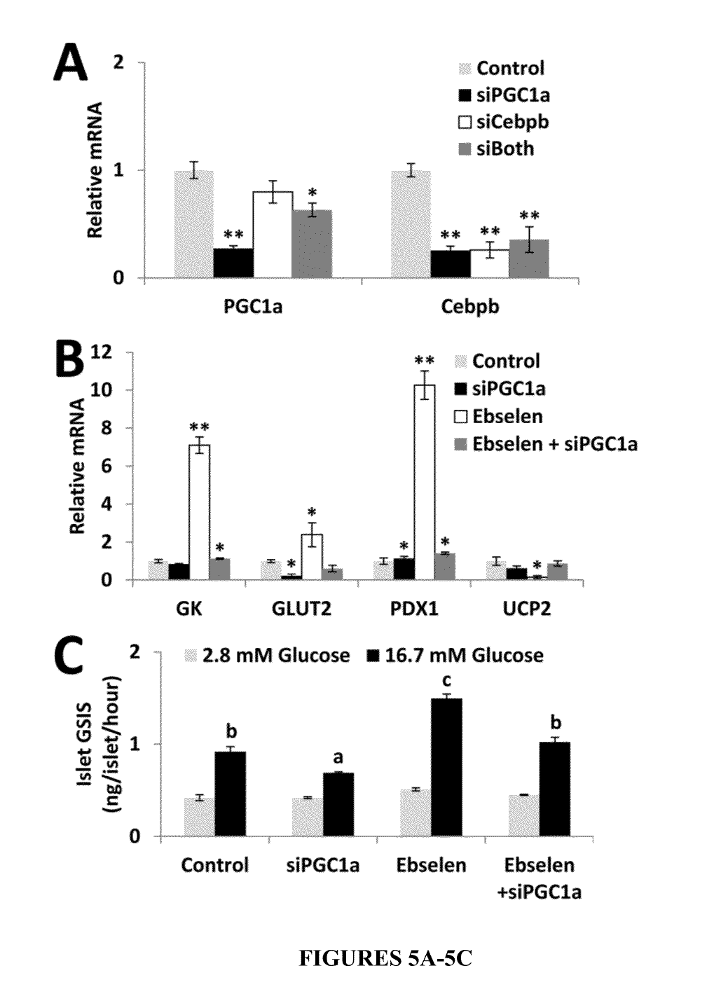

The overall positive responses of the ARE and GR pathway genes to the ebselen treatment indicated PGC-1α (the central effector of the ARE pathway) and C/EBPβ (the main effector of the GR pathway) as the primary mediators for the upstream signaling regulation of GSIS by ebselen. Subsequently, siRNA was applied to knock down these two genes in islets to assess their relative importance in the event. The pgc-1α siRNA suppressed gene expression of both pgc-1α and c/ebpβ by more than 70% (p<0.01), whereas the c/ebpβ siRNA decreased (p<0.01) only its own expression (FIG. 5A). The double siRNA did not produce suppression of either gene further than the single treatment. Most striking, knockdown of pgc-1α blocked the ebselen-induced up-regulation of gk, glut2, and pdx1 mRNA levels, down-regulation of ucp2 mRNA level (FIG. 5B), and GSIS rescue (FIG. 5C) in the GKO islets.

Rescue of GSIS in the GKO Mice by Ebselen

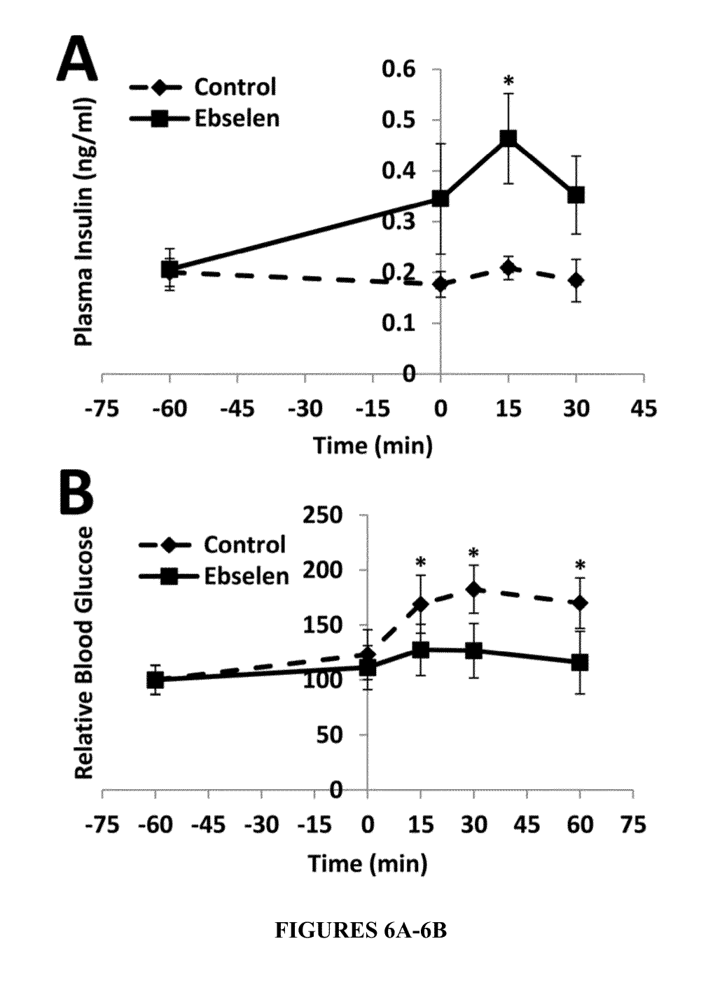

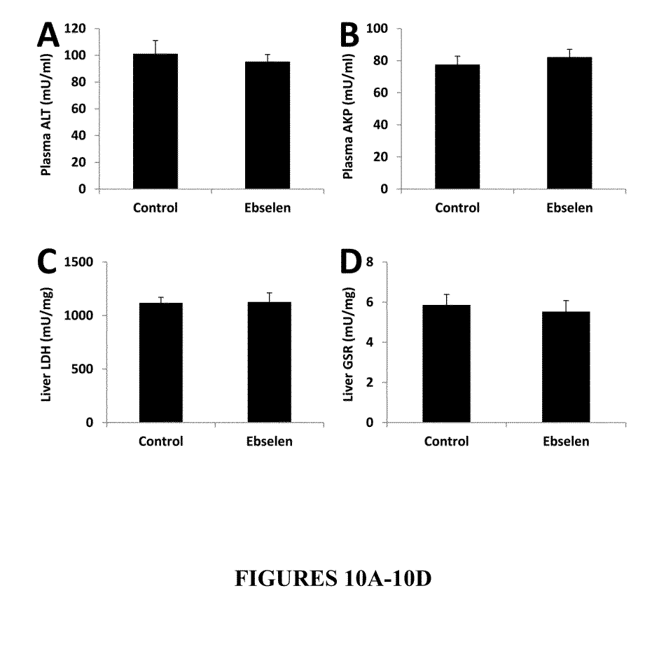

To determine if the observed rescue of GSIS in GKO islets by ebselen was reproducible at physiological conditions and if ebselen caused any “off-target” toxicity, the fasted GKO mice were given an i.p. injection of ebselen at 1 hour prior to the GSIS test. The injection elevated their plasma insulin concentrations at 0 min (baseline) by 95%, 15 min by 1.2-fold (p<0.05), and 30 min by 91% after the glucose challenge (FIG. 6A). Consequently, glucose clearance was improved by 17%, 18%, and 21% (p<0.05) at 15, 30, and 60 min, respectively, by the ebselen injection (FIG. 6B). The injection produced no differences from the control in activities of plasma alanine aminotransferase (ALT) and alkaline phosphatase (AKP) or hepatic LDH and GSR (FIGS. 10A-D).

### Example 10

### Discussion of Examples 1-9

The most exciting, novel finding of the present study was that the GPX mimic ebselen (Sies, H., “Ebselen, A Selenoorganic Compound as Glutathione Peroxidase Mimic,” Free Radic. Biol. Med. 14:313-323 (1993), which is hereby incorporated by reference in its entirety), at a relatively low dose (Costa et al., “Ebselen Reduces Hyperglycemia Temporarily-Induced by Diazinon: A Compound with Insulin-Mimetic Properties,” Chem. Biol. Interact. 197:80-86 (2012), which is hereby incorporated by reference in its entirety), rescued GSIS in the GKO islets and mice. Despite its recognition as the GPX mimic in 1984 (Muller et al., “A Novel Biologically Active Seleno-Organic Compound-I. Glutathione Peroxidase-Like Activity In Vitro and Antioxidant Capacity of PZ 51 (Ebselen),” Biochem. Pharmacol. 33:3235-3239 (1984), which is hereby incorporated by reference in its entirety) and subsequent extensive research on its protections against ROS, ischemic damage, and inflammation (Day, B. J., “Catalase and Glutathione Peroxidase Mimics. Biochem. Pharmacol. 77:285-296 (2009), which is hereby incorporated by reference in its entirety), only a couple of studies have explored its involvement in insulin synthesis (de-Mello et al., “Ebselen and Cytokine-Induced Nitric Oxide Synthase Expression in Insulin-Producing Cells,” Biochem. Pharmacol. 52:1703-1709 (1996), which is hereby incorporated by reference in its entirety) or hypoglycemic effect (Costa et al., “Ebselen Reduces Hyperglycemia Temporarily-Induced by Diazinon: A Compound with Insulin-Mimetic Properties,” Chem. Biol. Interact. 197:80-86 (2012), which is hereby incorporated by reference in its entirety). Unprecedentedly, the experiments described herein provide the direct evidence for a novel metabolic effect of ebselen in promoting GSIS. Because of the recently-discovered association between the GPX1 mutation and increased risk of type 2 diabetes (Ramprasath et al., “Genetic Association of Glutathione Peroxidase-1 (GPx-1) and NAD(P)H:Quinone Oxidoreductase 1 (NQ01) Variants and Their Association of CAD in Patients with Type-2 Diabetes,” Mol. Cell. Biochem. 361:143-150 (2012), which is hereby incorporated by reference in its entirety), applicant's findings offer a potentially new therapy of insulin secretion defects associated with diabetes and hyperglycemia (Lubos et al., “Glutathione Peroxidase-1 in Health and Disease: From Molecular Mechanisms to Therapeutic Opportunities,” Antioxid. Redox Signal. 15:1957-1997 (2011), which is hereby incorporated by reference in its entirety). Despite its lipophilic property, ebselen was prepared in 4% w/v hydroxypropyl-β-cyclodextrin for an i.p. injection to C57BL/6 mice (Singh et al., “A Safe Lithium Mimetic for Bipolar Disorder,” Nat. Commun. 4:1332 (2013), which is hereby incorporated by reference in its entirety) or was directly suspended in water for oral administration to humans (Yamaguchi et al., “Ebselen in Acute Ischemic Stroke: A Placebo-Controlled, Double-Blind Clinical Trial. Ebselen Study Group,” Stroke 29:12-17 (1998), which is hereby incorporated by reference in its entirety). The ebselen delivered in both ways was absorbed to blood and even crossed the blood-brain barrier (Singh et al., “A Safe Lithium Mimetic for Bipolar Disorder,” Nat. Commun. 4:1332 (2013); Yamaguchi et al., “Ebselen in Acute Ischemic Stroke: A Placebo-Controlled, Double-Blind Clinical Trial. Ebselen Study Group,” Stroke 29:12-17 (1998), which are hereby incorporated by reference in their entirety). Because ebselen is included in the National Institutes of Health Clinical Collection (Austin et al., “NIH Molecular Libraries Initiative,” Science 306:1138-1139 (2004), which is hereby incorporated by reference in its entirety), it has a history of use in human clinical trials and known safety profiles (www.nihchinicalcollection.com). Meanwhile, the ebselen doses used in the present study produced no “off-target” toxicity (Amacher, D. E., “A Toxicologist's Guide to Biomarkers of Hepatic Response,” Hum. Exp. Toxicol. 21:253-262 (2002), which is hereby incorporated by reference in its entirety) or side-effect on functions of thiol-containing enzymes (Lugokenski et al., “Inhibitory Effect of Ebselen on Lactate Dehydrogenase Activity from Mammals: A Comparative Study with Diphenyl Diselenide and Diphenyl Ditelluride,” Drug Chem. Toxicol. 34:66-76 (2011), which is hereby incorporated by reference in its entirety) in islets or liver. However, the long-term effectiveness of ebselen in promoting GSIS and the potential risk of over-stimulation (McClung et al., “Development of Insulin Resistance and Obesity in Mice Overexpressing Cellular Glutathione Peroxidase,” Proc. Natl. Acad. Sci. USA 101:8852-8857 (2004), which is hereby incorporated by reference in its entirety) associated with the treatment should be checked under physiological conditions (Ponzani, P., “Long-Term Effectiveness and Safety of Liraglutide in Clinical Practice,” Minerva Endocrinol. 38:103-112 (2013), which is hereby incorporated by reference in its entirety). Possible cross-talk between the pancreas and other tissues induced by the ebselen treatment (Cabou et al., “GLP-1, the Gut-Brain, and Brain-Periphery Axes,” Rev. Diabet. Stud. 8:418-431 (2011), which is hereby incorporated by reference in its entirety), and its global effect on the whole body ROS status (Kohen et al., “Oxidation of Biological Systems: Oxidative Stress Phenomena, Antioxidants, Redox Reactions, and Methods for Their Quantification,” Toxicol. Pathol. 30:620-650 (2002), which is hereby incorporated by reference in its entirety) should also be evaluated.

Ebselen rescued GSIS in the GKO islets by up-regulating GK, GLUT2, and PDX1, and down-regulating UCP2. Two strong forms of evidence showed that this rescue was executed by ebselen via ROS scavenging instead of simply being a Se carrier. The first evidence was the 74% decrease of intracellular ROS level in the ebselen-treated GKO islets compared with the control. The second evidence was the different effects of sodium selenite and ebselen on the four GSIS regulators or GSIS itself in the GKO islets. In fact, potential of ebselen as a Se carrier or transporter was questioned by a recent study due to the lack of stimulation of GPX activity or selenoprotein P expression in HepG2 cells (Hoefig et al., “Comparison of Different Selenocompounds with Respect to Nutritional Value vs. Toxicity Using Liver Cells in Culture,” J. Nutr. Biochem. 22:945-955 (2011), which is hereby incorporated by reference in its entirety). By taking four consecutive steps of genomics and bioinformatics analyses, it was revealed that the regulation of ebselen on the four key regulators of GSIS took place at transcription and was mediated by PGC-1α via the ARE and(or) GR pathways. In the first step, the Q-PCR analysis depicted parallel responses of mRNA and protein levels of GK, GLUT2, PDX1, and UCP2 in the GKO islets to ebselen, and suggested transcriptional regulation as the action site of ebselen. In the second step, four signal pathways (ARE, GR, Wnt, and NFAT) were identified as the potential mediator for the initial action of ebselen by analyzing gene promoters of the four key regulators of GSIS. Indeed, these pathways are highly responsive to redox regulation (Kao et al., “Glycyrrhizic Acid and 18beta-Glycyrrhetinic Acid Recover Glucocorticoid Resistance via PI3K-Induced API, CRE and NFAT Activation,” Phytomedicine 20:295-302 (2013); Ke et al., “Essential Role of ROS-Mediated NFAT Activation in TNF-Alpha Induction by Crystalline Silica Exposure,” Am. J. Physiol. Lung Cell. Mol. Physiol. 291:L257-L264 (2006); Tkachev et al., “Mechanism of the Nrf2/Keap1/ARE Signaling System,” Biochemistry (Mosc.) 76:407-422 (2011); Wen et al., “Reactive Oxygen Species and Wnt Signalling Crosstalk Patterns Mouse Extraembryonic Endoderm,” Cell. Signal. 24:2337-2348 (2012), which are hereby incorporated by reference in their entirety) and involved in transcriptional regulation of the key regulators of GSIS (Bordonaro, M., “Role of Wnt Signaling in the Development of Type 2 Diabetes,” Vitam. Horm. 80:563-581 (2009); Heit et al., “Calcineurin/NFAT Signalling Regulates Pancreatic Beta-Cell Growth and Function,” Nature 443:345-349 (2006); Soyal et al., “PGC-1 Alpha: A Potent Transcriptional Cofactor Involved in the Pathogenesis of Type 2 Diabetes,” Diabetologia 49:1477-1488 (2006); van Raalte et al., “Glucocorticoid Receptor Gene Polymorphisms are Associated with Reduced First-Phase Glucose-Stimulated Insulin Secretion and Disposition Index in Women, but not in Men,” Diabet. Med. 29:e211-e216 (2012), which are hereby incorporated by reference in their entirety). In the third step, the candidate pathways (ARE and GR) and mediators (PGC-1α and C/EBPβ) were chosen based on their gene expression responsiveness to ebselen. In the final step, siRNA was applied to prove that PGC-1α indeed served as the main mediator to link the ebselen-initiated intracellular ROS decrease to the downstream gene expression of gk, glut2, pdx1, and ucp2 for the GSIS rescue in the GKO islets.

There is both scientific and clinical significance to elucidate the novel role of PGC-1a in initiating the positive effect of the GPX mimic ebselen on GSIS. This reveals not only a new signal pathway to explain how GPX1 and(or) ebselen regulate insulin secretion, but also a new potential therapeutic target to treat insulin secretion disorders (Henquin, J. C., “Pathways in Beta-Cell Stimulus-Secretion Coupling as Targets for Therapeutic Insulin Secretagogues,” Diabetes 53(Suppl 3):S48-58 (2004), which is hereby incorporated by reference in its entirety). Expression and function of PGC-1α is highly responsive to ROS (Tkachev et al., “Mechanism of the Nrf2/Keap1/ARE Signaling System,” Biochemistry (Mosc.) 76:407-422 (2011), which is hereby incorporated by reference in its entirety). Down-regulation of PGC-1α decreased GSIS and(or) insulin production in both human and rat islets (Ling et al., “Epigenetic Regulation of PPARGC1A in Human Type 2 Diabetic Islets and Effect on Insulin Secretion,” Diabetologia 51:615-622 (2008), which is hereby incorporated by reference in its entirety). Two of its gene variants were associated with increased risks of type 2 diabetes in the Indian population (Bhat et al., “PGC-1 alpha Thr394Thr and Gly482Ser Variants are Significantly Associated with T2DM in Two North Indian Populations: A Replicate Case-Control Study,” Hum. Genet. 121:609-614 (2007); Yang et al., “Association of Peroxisome Proliferator-Activated Receptor Gamma Coactivator 1 Alpha (PPARGC1A) Gene Polymorphisms and Type 2 Diabetes Mellitus: A Meta-Analysis,” Diabetes Metab. Res. Rev. 27:177-184 (2011), which are hereby incorporated by reference in their entirety). As a transcriptional co-activator for PPARs and LXR (Puigserver et al., “Peroxisome Proliferator-Activated Receptor-Gamma Coactivator 1 Alpha (PGC-1 alpha): Transcriptional Coactivator and Metabolic Regulator,” Endocr. Rev. 24:78-90 (2003), which is hereby incorporated by reference in its entirety), it may play a dual role in GSIS upon the metabolic conditions (Gremlich et al., “Pancreatic Islet Adaptation to Fasting is Dependent on Peroxisome Proliferator-Activated Receptor Alpha Transcriptional Up-Regulation of Fatty Acid Oxidation,” Endocrinology 146:375-382 (2005); Helleboid-Chapman et al., “Glucose Regulates LXRalpha Subcellular Localization and Function in Rat Pancreatic Beta-Cells,” Cell Res. 16:661-670 (2006); Tordjman et al., “PPARalpha Suppresses Insulin Secretion and Induces UCP2 in Insulinoma Cells,” J. Lipid Res. 43:936-943 (2002), which are hereby incorporated by reference in their entirety). It is also interesting to note that PGC-1α activates gene expression of GPX1 and several other antioxidant enzymes (St-Pierre et al., “Suppression of Reactive Oxygen Species and Neurodegeneration by the PGC-1 Transcriptional Coactivators,” Cell 127:397-408 (2006), which is hereby incorporated by reference in its entirety). Thus, the ebselen-mediated activation of PGC-1α (Xiao et al., “Induction of Phase II Enzyme Activity by Various Selenium Compounds,” Nutr. Cancer 55:210-223 (2006), which is hereby incorporated by reference in its entirety) may constitute positive feedback between GPX1 and PGC-1α. However, potential roles of other redox-sensitive or ARE-related factors such as Nrf2 (and Nrf1) (Tkachev et al., “Mechanism of the Nrf2/Keap1/ARE Signaling System,” Biochemistry (Mosc.) 76:407-422 (2011), which is hereby incorporated by reference in its entirety) in the ebselen-induced cascade events should also be considered. In fact, Nrf2 was a downstream target molecule of PGC-1α in the pathway scheme derived from the present study, and its mRNA was up-regulated by ebselen in the GKO islets. Thus, roles of these two proteins were cooperative or coordinated in activating the cascade. In the future, plasmid reporter assays and/or chromatin immune-precipitation analyses shall be conducted to discern true initiation of transcription of GK, GLUT2, PDX1, and UCP2 by PGC-1α, Nrf2, and other factors in the ebselen-mediated GSIS rescue.

Another significant finding of the present study was that the GPX and SOD mimics demonstrated distinctly different regulation of signal transduction and gene expression related to GSIS. For many years, many antioxidants like these mimics have been perceived to be the same “pathway” or “family” members for ROS scavenging (Bisbal et al., “Antioxidants and Glucose Metabolism Disorders,” Curr. Opin. Clin. Nutr. Metab. Care 13:439-446 (2010), which is hereby incorporated by reference in its entirety). Using the GKO, SKO, and dKO islets, the intracellular H₂O₂ and superoxide status were metabolically controlled (Wang et al., “Knockouts of SOD1 and GPX1 Exert Different Impacts on Murine Islet Function and Pancreatic Integrity,” Antioxid. Redox Signal. 14:391-401 (2011), which is hereby incorporated by reference in its entirety) without confounding factors as encountered in conventional models (Lei et al., “Mice Deficient in Cu,Zn-Superoxide Dismutase are Resistant to Acetaminophen Toxicity,” Biochem. J. 399:455-461 (2006); McClung et al., “Development of Insulin Resistance and Obesity in Mice Overexpressing Cellular Glutathione Peroxidase,” Proc. Natl. Acad. Sci. USA 101:8852-8857 (2004); Wang et al., “Knockout of SOD1 Alters Murine Hepatic Glycolysis, Gluconeogenesis, and Lipogenesis,” Free Radic. Biol. Med. 53:1689-1696 (2012), which are hereby incorporated by reference in their entirety), for comparing functions and mechanisms of the GPX and SOD mimics in regulating islet GSIS. Overall, these two mimics depicted at least three distinctly different features. First, ebselen promoted GSIS in all four genotypes whereas CuDIPs helped only SKO islets. Second, ebselen caused consistent up-regulation of GK and GLUT2 and down-regulation of UCP2 in islets of the four genotypes, while CuDIPs produced an opposite effect on UCP2 between the WT and SKO islets. As an uncoupler of respiration and oxidative phosphorylation, UCP2 is activated by endogenously generated superoxide (Krauss et al., “Superoxide-Mediated Activation of Uncoupling Protein 2 Causes Pancreatic Beta Cell Dysfunction,” J. Clin. Invest. 112:1831-1842 (2003), which is hereby incorporated by reference in its entirety) and is a negative regulator of insulin secretion in β-cells (Zhang et al., “Uncoupling Protein-2 Negatively Regulates Insulin Secretion and is a Major Link Between Obesity, Beta Cell Dysfunction, and Type 2 Diabetes,” Cell 105:745-755 (2001), which is hereby incorporated by reference in its entirety). Thus, down-regulation of UCP2 in all genotypes by ebselen and in SKO by CuDIPs was consistent with their effects on GSIS. However, these two mimics exhibited an opposite effect on UCP2 in the WT islets (CuDIPs showed no effect on GSIS in the WT islets). Lastly, gene expression of the four identified pathways related to GSIS was largely suppressed by CuDIPs, but promoted by ebselen in the dKO islets. This helps explain why ebselen but not CuDIPs promoted GSIS in the dKO islets. Comparatively, the impact of ebselen and CuDIPs on islet GSIS and related pathways was in line with those of GKO and SKO on hepatotoxicity of acetaminophen (Lei et al., “Mice Deficient in Cu,Zn-Superoxide Dismutase are Resistant to Acetaminophen Toxicity,” Biochem. J. 399:455-461 (2006), which is hereby incorporated by reference in its entirety), femoral mechanical characteristics (Wang et al., “Knockouts of Se-Glutathione Peroxidase-1 and Cu,Zn Superoxide Dismutase Exert Different Impacts on Femoral Mechanical Performance of Growing Mice,” Mol. Nutr. Food Res. 52:1334-1339 (2008), which is hereby incorporated by reference in its entirety), islet β cell mass and insulin synthesis (Wang et al., “Knockouts of SOD1 and GPX1 Exert Different Impacts on Murine Islet Function and Pancreatic Integrity,” Antioxid. Redox Signal. 14:391-401 (2011), which is hereby incorporated by reference in its entirety), and hepatic energy metabolism (Wang et al., “Knockout of SOD1 Alters Murine Hepatic Glycolysis, Gluconeogenesis, and Lipogenesis,” Free Radic. Biol. Med. 53:1689-1696 (2012), which is hereby incorporated by reference in its entirety). Although antioxidants are often perceived to be beneficial to islet β-cell function and survival, clinical applications remain controversial or restricted to a narrow window of therapeutic dosage (Bisbal et al., “Antioxidants and Glucose Metabolism Disorders,” Curr. Opin. Clin. Nutr. Metab. Care 13:439-446 (2010), which is hereby incorporated by reference in its entirety). Our finding highlights the importance of discretionary use of antioxidants in clinical treatment of GSIS to avoid harmful effects based on reciprocal results of seemingly “similar” antioxidant compounds. Furthermore, altering GPX1 and SOD1 activities might be applied to manipulate or restore intracellular H₂O₂ and superoxide balance in islets as a new or more effective strategy to treat insulin secretion disorders, in comparison with the insulin-centric therapy of insulin stimulators or analogues (Robertson, R. P., “Antioxidant Drugs for Treating Beta-Cell Oxidative Stress in Type 2 Diabetes: Glucose-Centric Versus Insulin-Centric Therapy,” Discov. Med. 9:132-137 (2010), which is hereby incorporated by reference in its entirety).

However, it seems puzzling that the increased H₂O₂ by GKO impaired GSIS and the removal of H₂O₂ by ebselen increased GSIS, whereas the block of enzymatic H₂O₂ production from superoxide by SKO also impaired GSIS and the recovery of H₂O₂ production by CuDIPs in the SKO islets rescued GSIS. This again underscores the complexity of the ROS regulation on GSIS, and may be explained by the concentration-dependent dual effect of H₂O₂ (Iwakami et al., “Concentration-Dependent Dual Effects of Hydrogen Peroxide on Insulin Signal Transduction in H4IIEC Hepatocytes,” PLoS One 6:e27401 (2011), which is hereby incorporated by reference in its entirety). Whereas excessive H₂O₂ could suppress GSIS (Pi et al., “ROS Signaling, Oxidative Stress and Nrf2 in Pancreatic Beta-Cell Function,” Toxicol. Appl. Pharmacol. 244:77-83 (2009), which is hereby incorporated by reference in its entirety), a minimal amount of H₂O₂ from the glucose metabolism is an essential signal molecule for triggering GSIS (Pi et al., “Reactive Oxygen Species as a Signal in Glucose-Stimulated Insulin Secretion,” Diabetes 56:1783-1791 (2007), which is hereby incorporated by reference in its entirety). Thus, the SKO islets might lack sufficient H₂O₂ or appropriate ratios of H₂O₂ to superoxide to initiate GSIS, and the CuDIPs treatment rescued this function by restoring H₂O₂ generation. However, this notion could not explain the positive effect of ebselen on GSIS in the SKO islets. Likewise, sodium selenite improved GSIS in the SKO and dKO islets, but not in the WT or GKO islets. This might be due to a low SOD1-like catalytic activity of selenite in transforming superoxide to H₂O₂ (Feroci et al., “Study of the Antioxidant Effect of Several Selenium and Sulphur Compounds,” J. Trace Elem. Med. Biol. 12:96-100 (1998), which is hereby incorporated by reference in its entirety) in the SKO and dkO islets. However, selenite did not affect GSIS of the WT and GKO islets, which was different from that reported in rat islets and min6 cells (Campbell et al., “Selenium Stimulates Pancreatic Beta-Cell Gene Expression and Enhances Islet Function,” FEBS Lett. 582:2333-2337 (2008), which is hereby incorporated by reference in its entirety). Because Gpx1 and ebselen were supposed to reduce both H₂O₂ and organic hydroperoxides (Maiorino et al., “Kinetic Mechanism and Substrate Specificity of Glutathione Peroxidase Activity of Ebselen (PZ51),” Biochem. Pharmacol. 37:2267-2271 (1988), which is hereby incorporated by reference in its entirety), the latter were likely involved in the impaired GSIS in GKO mice and the rescue by ebselen. Although lipid profiles (total cholesterol, total triglyceride, and nonesterified fatty acid) were not different between the GKO and WT mice (Wang et al., “Knockout of SOD1 Alters Murine Hepatic Glycolysis, Gluconeogenesis, and Lipogenesis,” Free Radic. Biol. Med. 53:1689-1696 (2012), which is hereby incorporated by reference in its entirety), the role of organic hydroperoxides in the ebselen-rescued GSIS needs further research. In addition, ebselen stimulated insulin secretion in the WT and GKO islets at 2.8 mM glucose, and CUMIN did that in the SKO islets. Although low glucose level at 2.8 mM often inhibits insulin secretion by other stimuli, e.g. glucagon-like peptide 1 (“GLP-1”) and glucose-dependent insulinotropic polypeptide (“GIP”) (Gromada et al., “Cellular Regulation of Islet Hormone Secretion by the Incretin Hormone Glucagon-Like Peptide 1,” Pflugers Arch. 435:583-594 (1998), which is hereby incorporated by reference in its entirety), certain therapeutic insulin secretagogues such as glibenclamide elevated islet insulin secretion at both basal (1 mM) and high (15 mM) glucose levels (Henquin, J. C., “Pathways in Beta-Cell Stimulus-Secretion Coupling as Targets for Therapeutic Insulin Secretagogues,” Diabetes 53(Suppl 3):548-58 (2004), which is hereby incorporated by reference in its entirety). Likewise, GK activators also increased beta cell cytosolic calcium and insulin secretion at 1 mM glucose (Matschinsky, F. M., “Assessing the Potential of Glucokinase Activators in Diabetes Therapy,” Nat. Rev. Drug Discov. 8:399-416 (2009), which is hereby incorporated by reference in its entirety), Despite, stimulation of insulin secretion by ebselen and. CuDIPs in the respective genotypes was still stronger at 16.7 than 2.8 mM glucose in the present study.

In summary, applicants have demonstrated a novel metabolic role and therapeutic potential of the GPX mimic ebselen (Sies, H., “Ebselen, A Selenoorganic Compound as Glutathione Peroxidase Mimic,” Free Radic. Biol. Med. 14:313-323 (1993), which is hereby incorporated by reference in its entirety) in rescuing GSIS in the GKO islets and mice. This rescue constituted coordinated regulation of GK, GLUT2, PDX1, and UCP2 by activating the PGC-1α mediated ARE and(or) GR signaling. In comparison, the SOD mimic CuDIPs exerted different impacts on GSIS and the related gene expression. Thus, applicants' findings have clarified that GPX1 and SOD1, as two important intracellular antioxidant enzymes, function distinguishably in regulating insulin secretion. Most likely, evolution has selected unique signaling pathways for different antioxidant enzymes or compounds to precisely control insulin secretion in response to complicated metabolic conditions. Clinically, these unique features of different antioxidant enzymes or mimics may be used to fine-tune islet ROS status and the related signaling for effective treatments of insulin secretion and diabetic disorders.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of enhancing glucose-stimulated insulin secretion in a subject, said method comprising:
selecting a subject with: (1) an antioxidant deficiency and (2) a need for enhanced glucose-stimulated insulin secretion and
administering to the selected subject an agent selected from the group consisting of (1) a compound according to Formula I or a pharmaceutically acceptable salt thereof: 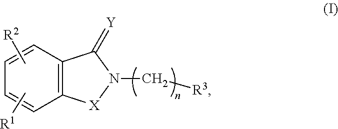
wherein
R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;
R³ is aryl optionally substituted with R⁴;
R⁴ is selected from the group consisting of H, halogen, —OH, —CF₃, —NO₂, —NR⁵R⁶, C₁-C₆ alkyl, and C₁-C₆ alkoxyl;
R⁵ and R⁶ are independently selected from the group consisting of H and C₁-C₆ alkyl;
X is Se or S;
Y is O or S; and
n is 0 to 5, (2) glutathione peroxidase, or (3) activators of PGC-1α antioxidant response element, under conditions effective to enhance glucose-stimulated insulin secretion in the subject.

2. The method of claim 1, wherein the agent is a compound according to Formula I or a pharmaceutically acceptable salt thereof: 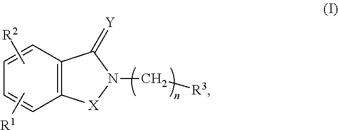
wherein
R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;
R³ is aryl optionally substituted with R⁴;
R⁴ is selected from the group consisting of H, halogen, —OH, —CF₃, —NO₂, —NR⁵R⁶, C₁-C₆ alkyl, and C₁-C₆ alkoxyl;
R⁵ and R⁶ are independently selected from the group consisting of H and C₁-C₆ alkyl;
X is Se or S;
Y is O or S; and
n is 0 to 5.

3. The method of claim 2, wherein n is 0, R¹ is H, R² is H, R³ is phenyl, X is Se, and Y is O.

4. The method of claim 1, wherein the agent is glutathione peroxidase.

5. The method of claim 1, wherein the agent is an activator of PGC-1α antioxidant response element.

6. The method according to claim 1, wherein the said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

7. A method of treating a subject with Type 2 diabetes, said method comprising:
selecting a subject with: (1) an antioxidant deficiency and (2) Type 2 diabetes and
administering to the selected subject an agent selected from the group consisting of (1) a compound according to Formula I or a pharmaceutically acceptable salt thereof: 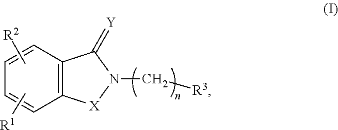
wherein
R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;
R³ is aryl optionally substituted with R⁴;
R⁴ is selected from the group consisting of H, halogen, —OH, —CF₃, —NO₂, —NR⁵R⁶, C₁-C₆ alkyl, and C₁-C₆ alkoxyl;
R⁵ and R⁶ are independently selected from the group consisting of H and C₁-C₆ alkyl;
X is Se or S;
Y is O or S; and
n is 0 to 5, (2) glutathione peroxidase, or (3) activators of PGC-1α antioxidant response element, under conditions effective to treat Type 2 diabetes in the subject.

8. The method according to claim 7, wherein the said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

9. The method of claim 7, wherein the agent is a compound according to Formula I or a pharmaceutically acceptable salt thereof: 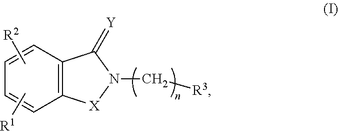
wherein
R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;
R³ is aryl optionally substituted with R⁴;
R⁴ is selected from the group consisting of H, halogen, —OH, —CF₃, —NO₂, —NR⁵R⁶, C₁-C₆ alkyl, and C₁-C₆ alkoxyl;
R⁵ and R⁶ are independently selected from the group consisting of H and C₁-C₆ alkyl;
X is Se or S;
Y is O or S; and
n is 0 to 5.

10. The method of claim 9, wherein n is 0, R¹ is H, R² is H, R³ is phenyl, X is Se, and Y is O.

11. The method of claim 7, wherein the agent is glutathione peroxidase.

12. The method of claim 7, wherein the agent is an activator of PGC-1α antioxidant response element.

13. A method of treating a subject with hypoglycemia, said method comprising:
selecting a subject with: (1) an antioxidant deficiency and (2) hypoglycemia, and
administering to the selected subject an agent selected from the group consisting of (1) a compound according to Formula I or a pharmaceutically acceptable salt thereof: 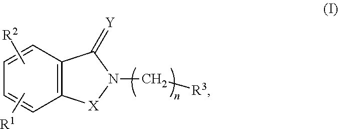
wherein
R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;
R³ is aryl optionally substituted with R⁴;
R⁴ is selected from the group consisting of H, halogen, —OH, —CF₃, —NO₂, —NR⁵R⁶, C₁-C₆ alkyl, and C₁-C₆ alkoxyl;
R⁵ and R⁶ are independently selected from the group consisting of H and C₁-C₆ alkyl;
X is Se or S;
Y is O or S; and
n is 0 to 5, (2) glutathione peroxidase, or (3) activators of PGC-1α antioxidant response element, under conditions effective to treat the subject with hypoglycemia.

14. The method according to claim 13, wherein the said administering is carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes.

15. The method of claim 13, wherein the agent is a compound according to Formula I or a pharmaceutically acceptable salt thereof: 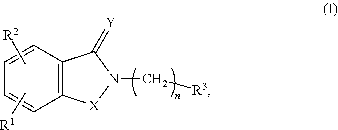
wherein
R¹ and R² are independently selected from the group consisting of H, halogen, —OH, —CF₃, —NO₂, —NR⁵R⁶, C₁-C₆ alkyl, and C₁-C₆ alkoxyl, or R¹ and R² may combine together to form a methylenedioxy group;
R³ is aryl optionally substituted with R⁴;
R⁴ is selected from the group consisting of H, halogen, —OH, —CF₃, —NO₂, —NR⁵R⁶, C₁-C₆ alkyl, and C₁-C₆ alkoxyl;
R⁵ and R⁶ are independently selected from the group consisting of H and C₁-C₆ alkyl;
X is Se or S;
Y is O or S; and
n is 0 to 5.

16. The method of claim 15, wherein n is 0, R¹ is H, R² is H, R³ is phenyl, X is Se, and Y is O.

17. The method of claim 13, wherein the agent is glutathione peroxidase.

18. The method of claim 13, wherein the agent is an activator of PGC-1α antioxidant response element.


SEQUENCE LISTING (68 sequences)

SEQ ID NO: 1 - DNA, 25 bp, artificial
  Forward Primer
tttccctggttaagcagtacagccc

SEQ ID NO: 2 - DNA, 24 bp, artificial
  Reverse Primer
tggcctgtatccaacacttcgaga

SEQ ID NO: 3 - DNA, 24 bp, artificial
  Forward Primer
tgaggaccaacacaagctgtacca

SEQ ID NO: 4 - DNA, 24 bp, artificial
  Reverse Primer
tggtcagcatctggttgatgaggt

SEQ ID NO: 5 - DNA, 24 bp, artificial
  Forward Primer
acagcagattctagtgcccagcaa

SEQ ID NO: 6 - DNA, 24 bp, artificial
  Reverse Primer
tcttcagcaggctgtgtaggaagt

SEQ ID NO: 7 - DNA, 24 bp, artificial
  Forward Primer
cctttcagaaccaccaaagcggaa

SEQ ID NO: 8 - DNA, 24 bp, artificial
  Reverse Primer
aagtcaggaatcccacaggagaca

SEQ ID NO: 9 - DNA, 24 bp, artificial
  Forward Primer
agcactcagaaccatgcagcaaac

SEQ ID NO: 10 - DNA, 24 bp, artificial
  Reverse Primer
tttggtgtgaggagggtcatcgtt

SEQ ID NO: 11 - DNA, 24 bp, artificial
  Forward Primer
agtgatgtccgcacagaagagcaa

SEQ ID NO: 12 - DNA, 24 bp, artificial
  Reverse Primer
gtggcctgagtttgtgtttgctga

SEQ ID NO: 13 - DNA, 24 bp, artificial
  Forward Primer
aggttgcccacattcccaaacaag

SEQ ID NO: 14 - DNA, 24 bp, artificial
  Reverse Primer
tgaagactgaactttcagcgtggc

SEQ ID NO: 15 - DNA, 24 bp, artificial
  Forward Primer
aagagcgtgccctacttcaaggat

SEQ ID NO: 16 - DNA, 24 bp, artificial
  Reverse Primer
gtgaagggacagattgtggcgaat

SEQ ID NO: 17 - DNA, 24 bp, artificial
  Forward Primer
ttcggcatggccaagattgacaac

SEQ ID NO: 18 - DNA, 24 bp, artificial
  Reverse Primer
ttggtgcccatgtgttcttgcatc

SEQ ID NO: 19 - DNA, 24 bp, artificial
  Forward Primer
acataaagtccttcccgctgacca

SEQ ID NO: 20 - DNA, 24 bp, artificial
  Reverse Primer
aaattcggatggccacctctttgc

SEQ ID NO: 21 - DNA, 24 bp, artificial
  Forward Primer
gcagtgaaatgggcaaaggcgata

SEQ ID NO: 22 - DNA, 24 bp, artificial
  Reverse Primer
ccagggcaaatgccatgagaaaca

SEQ ID NO: 23 - DNA, 24 bp, artificial
  Forward Primer
tgtccacaggacagaagcgattga

SEQ ID NO: 24 - DNA, 24 bp, artificial
  Reverse Primer
atcttatgaacagcgtcgccaggt

SEQ ID NO: 25 - DNA, 24 bp, artificial
  Forward Primer
acaagctgagcgacgagtacaaga

SEQ ID NO: 26 - DNA, 23 bp, artificial
  Reverse Primer
gacagctgctccaccttcttctg

SEQ ID NO: 27 - DNA, 24 bp, artificial
  Forward Primer
ttcaggaacaggtggagaagctca

SEQ ID NO: 28 - DNA, 24 bp, artificial
  Reverse Primer
tttctcctgcaggctttcgatgtg

SEQ ID NO: 29 - DNA, 24 bp, artificial
  Forward Primer
tccaagagcaagtggagaagctca

SEQ ID NO: 30 - DNA, 24 bp, artificial
  Reverse Primer
actcttccaaagcacactgggact

SEQ ID NO: 31 - DNA, 24 bp, artificial
  Forward Primer
tgatggacgctcagatgcaaacct

SEQ ID NO: 32 - DNA, 24 bp, artificial
  Reverse Primer
agaaactctggaagctcacagcca

SEQ ID NO: 33 - DNA, 24 bp, artificial
  Forward Primer
tgacatgcagatggacaagacgga

SEQ ID NO: 34 - DNA, 24 bp, artificial
  Reverse Primer
tgcagtacgcttctagtgacgcat

SEQ ID NO: 35 - DNA, 24 bp, artificial
  Forward Primer
aggccgctactatgccatgaagat

SEQ ID NO: 36 - DNA, 24 bp, artificial
  Reverse Primer
tggaatgagtacttgagggccgta

SEQ ID NO: 37 - DNA, 24 bp, artificial
  Forward Primer
ttggagccactgattacacgtcca

SEQ ID NO: 38 - DNA, 24 bp, artificial
  Reverse Primer
ttccaccaactgatccacaccact

SEQ ID NO: 39 - DNA, 24 bp, artificial
  Forward Primer
tgggactctgcacaacctttctca

SEQ ID NO: 40 - DNA, 24 bp, artificial
  Reverse Primer
agtgtcgtgatggcgtagaacagt

SEQ ID NO: 41 - DNA, 24 bp, artificial
  Forward Primer
agagagtgcagccatcaaccagat

SEQ ID NO: 42 - DNA, 24 bp, artificial
  Reverse Primer
ctgcatgtgaagctgtcgttcctt

SEQ ID NO: 43 - DNA, 24 bp, artificial
  Forward Primer
tgagagccaaggtcattgctgagt

SEQ ID NO: 44 - DNA, 24 bp, artificial
  Reverse Primer
ttccttgcgggccagttcatagta

SEQ ID NO: 45 - DNA, 24 bp, artificial
  Forward Primer
aggagtttcactacagccggtaca

SEQ ID NO: 46 - DNA, 24 bp, artificial
  Reverse Primer
ctgctgctgctgctgtttcttctt

SEQ ID NO: 47 - DNA, 24 bp, artificial
  Forward Primer
atggtggctacagccagctatgaa

SEQ ID NO: 48 - DNA, 24 bp, artificial
  Reverse Primer
tcacccttccgtagctcaatgtct

SEQ ID NO: 49 - DNA, 24 bp, artificial
  Forward Primer
atgcccaatgtttccaacgacctg

SEQ ID NO: 50 - DNA, 24 bp, artificial
  Reverse Primer
gcccagcatgcagatgaatcacaa

SEQ ID NO: 51 - DNA, 24 bp, artificial
  Forward Primer
tgtgtacacggtggtttgtctcca

SEQ ID NO: 52 - DNA, 24 bp, artificial
  Reverse Primer
acagcctctgactgtgttgtgagt

SEQ ID NO: 53 - DNA, 21 bp, artificial
  Forward Primer
aagtgctctcctgctttccca

SEQ ID NO: 54 - DNA, 23 bp, artificial
  Reverse Primer
tttgtccagctccactgccttct

SEQ ID NO: 55 - DNA, 24 bp, artificial
  Forward Primer
ttcctttgaccctagccggacata

SEQ ID NO: 56 - DNA, 24 bp, artificial
  Reverse Primer
aacactggtgtctctcccaaaggt

SEQ ID NO: 57 - DNA, 24 bp, artificial
  Forward Primer
agggtgagcctgtatgtaatgcct

SEQ ID NO: 58 - DNA, 24 bp, artificial
  Reverse Primer
aggacctgctggcgtcttagattt

SEQ ID NO: 59 - DNA, 24 bp, artificial
  Forward Primer
agggaacaacatcgtgggacttct

SEQ ID NO: 60 - DNA, 24 bp, artificial
  Reverse Primer
gtgtcattcaccattgccaccaca

SEQ ID NO: 61 - DNA, 24 bp, artificial
  Forward Primer
aaaggaagaggcatcgactgagca

SEQ ID NO: 62 - DNA, 24 bp, artificial
  Reverse Primer
acaccaatggttgcatacacaggc

SEQ ID NO: 63 - DNA, 24 bp, artificial
  Forward Primer
agctccctttcccgtggatgaaat

SEQ ID NO: 64 - DNA, 24 bp, artificial
  Reverse Primer
taggcagtacgggtcctcttgttt

SEQ ID NO: 65 - DNA, 24 bp, artificial
  Forward Primer
agcctacaagaccattgcacgaga

SEQ ID NO: 66 - DNA, 24 bp, artificial
  Reverse Primer
ataggtcaccagctcagcacagtt

SEQ ID NO: 67 - DNA, 24 bp, artificial
  Forward Primer
taaagctggtgggcatccagtaac

SEQ ID NO: 68 - DNA, 24 bp, artificial
  Reverse Primer
gggaccacaaagatgctgtttgac